(19) United States
(12) Patent Application Publication
(10) Pub. No.: US 2010/0179208 A1
(43) Pub. Date: Jul. 15, 2010

(54) Use of HDAC Inhibitors for the Treatment of Bone Destruction

(75) Inventors: Peter Wisdom Atadja, Acton, MA (US); Joseph Daniel Growney, Reading, MA (US); Wenlin Shao, Dedham, MA (US)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, BASEL (CH)

(21) Appl. No.: 12/601,538

(22) PCT Filed: May 28, 2008
(86) PCT No.: PCT/US08/64944

§ 371 Date: Nov. 24, 2009

Related U.S. Application Data
(60) Provisional application No. 60/940,735, filed on May 30, 2007.

(51) Int. Cl. A61K 31/405; A61P 19/10

(52) U.S. Cl. 514/415

(57) ABSTRACT

The present invention relates to the use of HDAC inhibitors for the treatment of bone destruction caused by cancer, inflammatory diseases and osteoporosis.

### FIELD OF THE INVENTION

The invention relates to the use of an histone deacetylase (HDAC) inhibitor or a pharmaceutically acceptable salt thereof for the manufacture of pharmaceutical compositions for the treatment of bone destruction associated with cancer, inflammatory diseases and osteoporosis; the use of an HDAC inhibitor or a pharmaceutically acceptable salt thereof in the treatment of bone destruction associated with cancer, inflammatory diseases and osteoporosis; a method of treating warm-blooded animals including mammals, especially humans, suffering from of bone destruction associated with cancer, inflammatory diseases and osteoporosis by administering to a said animal in need of such treatment a dose effective against said disease of an HDAC inhibitor or a pharmaceutically acceptable salt thereof.

### BACKGROUND OF THE INVENTION

The normal bone turnover is regulated by the balance between the osteolytic activity of osteoclasts and the bone forming activity of osteoblasts. Bone integrity may be compromised in patients suffering from cancer, inflammatory diseases and osteoporosis. Therefore, there is a need to develop novel treatment methods using HDAC inhibitors.

### SUMMARY OF THE INVENTION

The compounds as defined herein, are HDAC inhibitors. Reversible acetylation of histones is a major regulator of gene expression that acts by altering accessibility of transcription factors to DNA. In normal cells, deacetylase (HDA) and histone acetyltrasferase together control the level of acetylation of histones to maintain a balance. Inhibition of HDA results in the accumulation of hyperacetylated histones, which results in a variety of cellular responses.

Surprisingly, it was now found that HDAC inhibitors, especially the compounds of formula (I), as defined herein, treat bone destruction associated with cancer. More specifically the cancer is multiple myeloma, breast cancer or prostate cancer. Hence, the invention relates to the use of an HDAC inhibitor for the preparation of a medicament for the treatment of bone destruction associated with cancer. The invention also relates to the use of an HDAC inhibitor or a pharmaceutically acceptable salt thereof in the treatment of bone destruction associated with cancer. The invention relates to a method of treating warm-blooded animals including mammals, especially humans, suffering from bone destruction associated with cancer by administering to a said animal in need of such treatment a dose effective against said disease of an HDAC inhibitor or a pharmaceutically acceptable salt thereof.

Surprisingly, it was now found that HDAC inhibitors, especially the compounds of formula (I), as defined herein, treat bone destruction associated with inflammatory diseases. Hence, the invention relates to the use of an HDAC inhibitor for the preparation of a medicament for the treatment of bone destruction associated with inflammatory diseases. The invention also relates to the use of an HDAC inhibitor or a pharmaceutically acceptable salt thereof in the treatment of bone destruction associated with inflammatory diseases. The invention relates to a method of treating warm-blooded animals including mammals, especially humans, suffering from bone destruction associated with inflammatory diseases by administering to a said animal in need of such treatment a dose effective against said disease of an HDAC inhibitor or a pharmaceutically acceptable salt thereof.

Surprisingly, it was now found that HDAC inhibitors, especially the compounds of formula (I), as defined herein, treat bone destruction associated with osteoporosis. Hence, the invention relates to the use of an HDAC inhibitor for the preparation of a medicament for the treatment of bone destruction associated with osteoporosis. The invention also relates to the use of an HDAC inhibitor or a pharmaceutically acceptable salt thereof in the treatment of bone destruction associated with osteoporosis. The invention relates to a method of treating warm-blooded animals including mammals, especially humans, suffering from bone destruction associated with osteoporosis by administering to a said animal in need of such treatment a dose effective against said disease of an HDAC inhibitor or a pharmaceutically acceptable salt thereof.

### DETAILED DESCRIPTION OF THE FIGURES

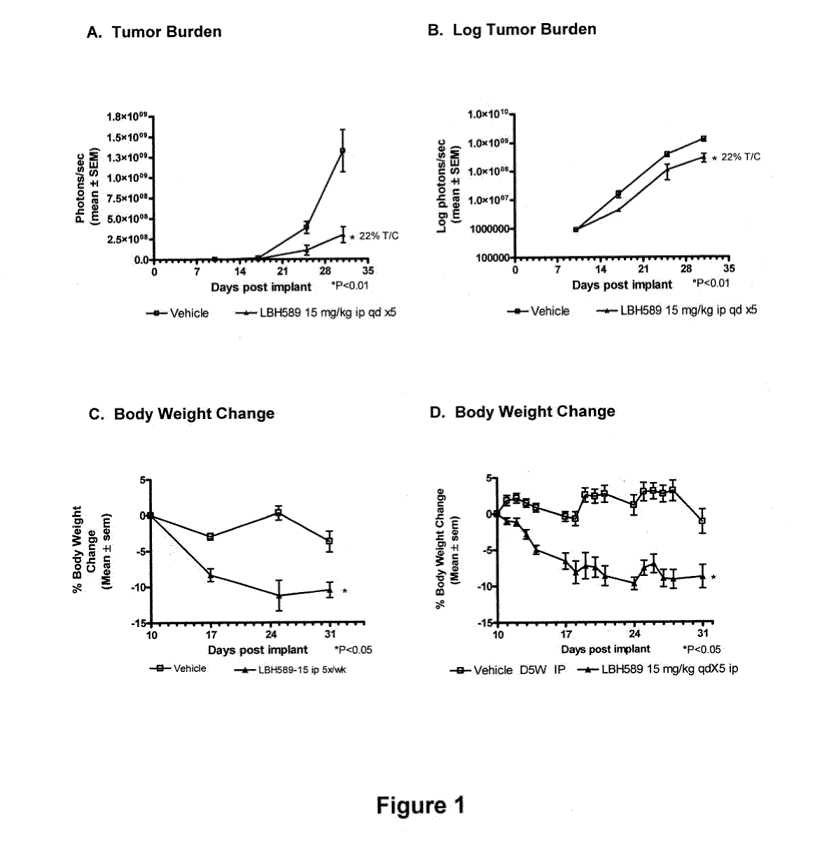

FIG. 1 illustrates LBH589 effects on tumor burden and body Weight in study #0879.

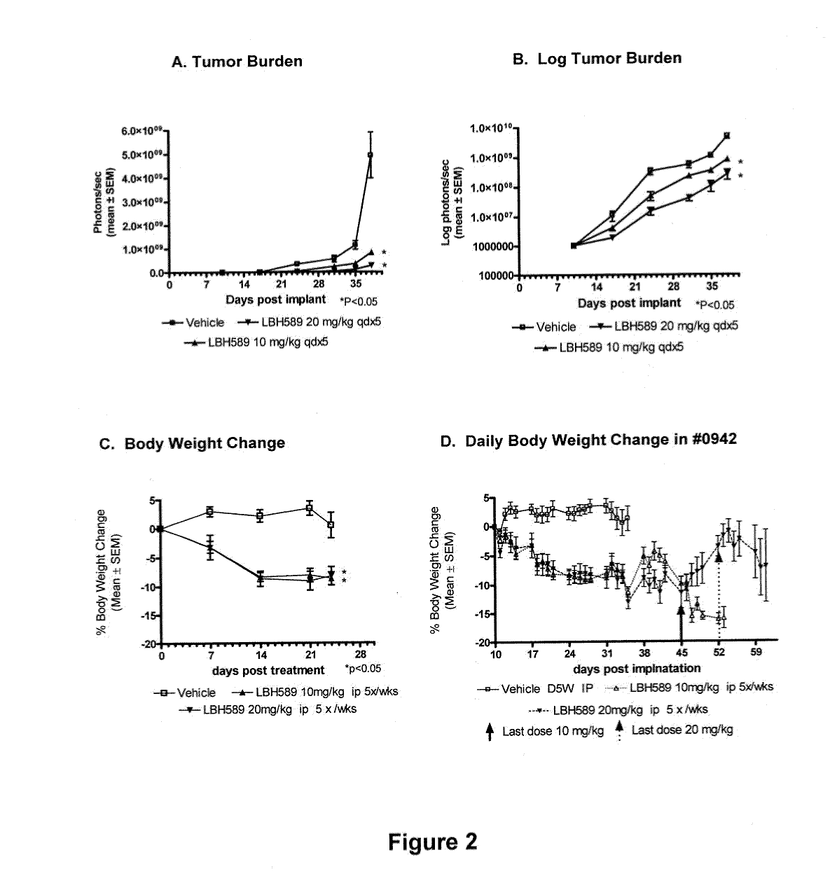

FIG. 2 illustrates LBH589 effects on tumor burden and body Weight in study #0942.

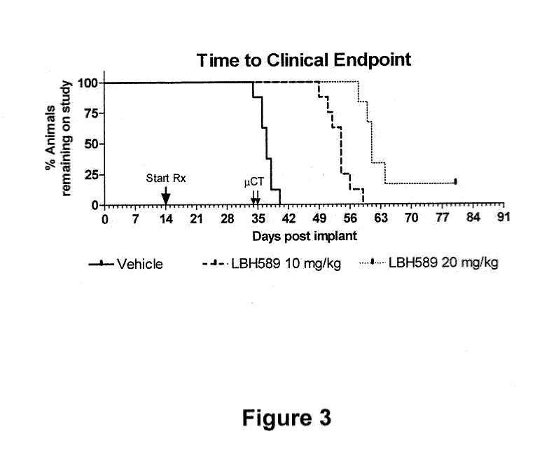

FIG. 3 illustrates LBH589 effects on time to clinical endpoint in #0942.

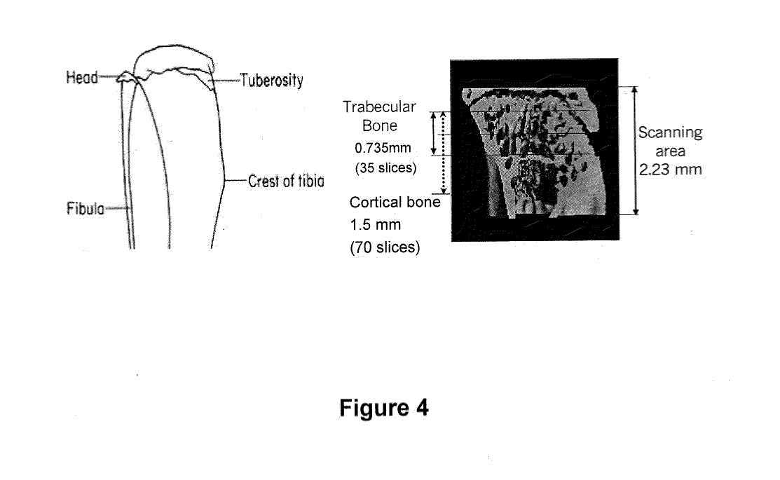

FIG. 4 illustrates MicroCT scanning and trabecular bone measurement region of interest.

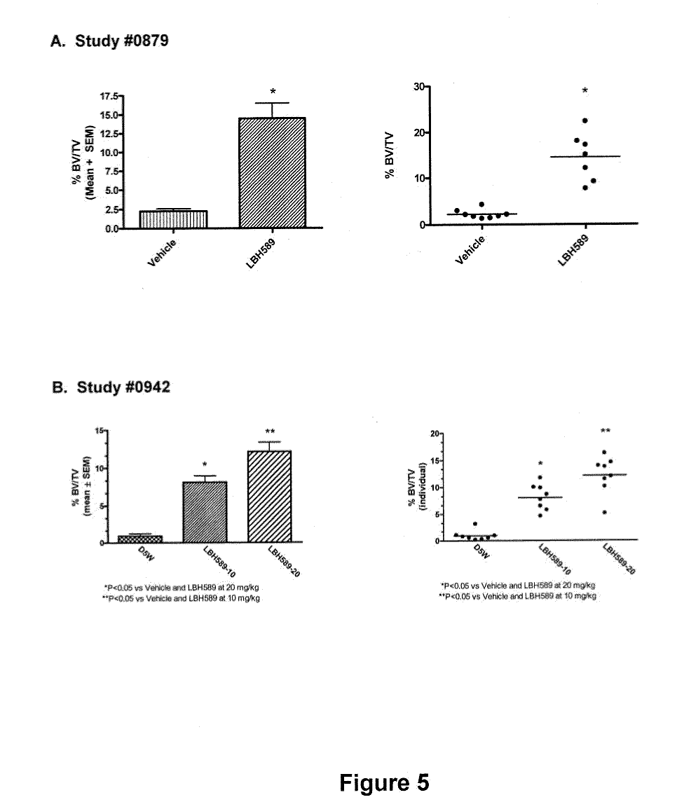

FIG. 5 describes LBH589 effects on tibial trabecular bone in Study #879 and #0942.

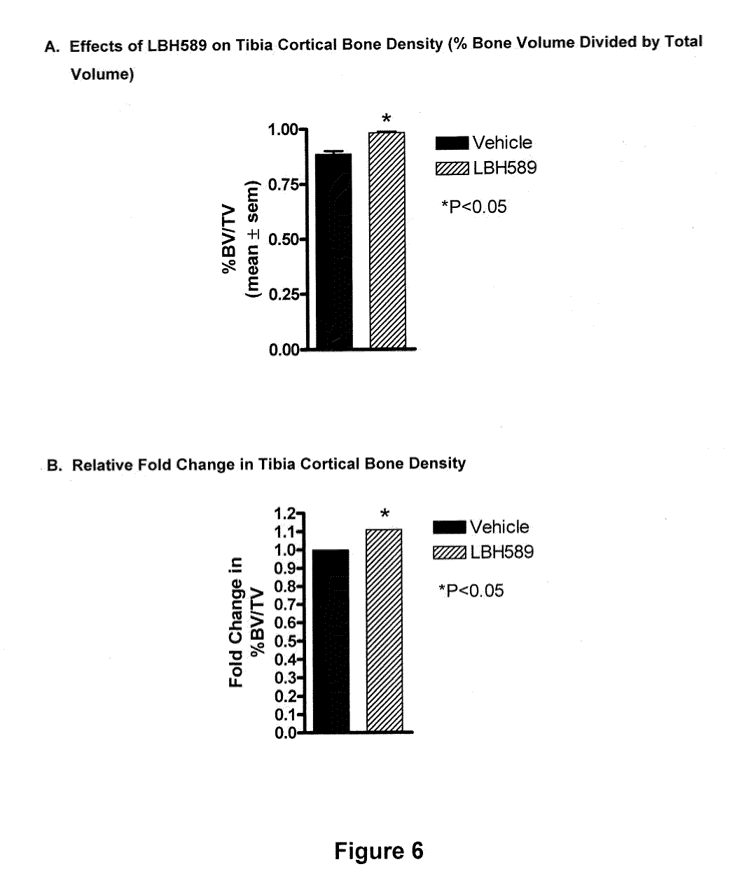

FIG. 6 describes LBH589 effects on tibial cortical bone.

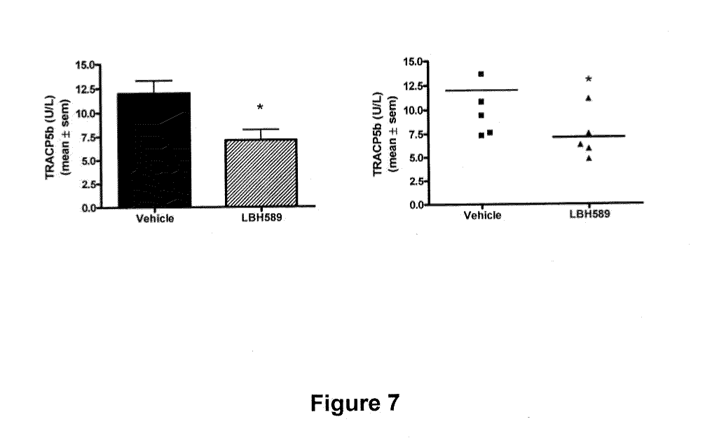

FIG. 7 describes LBH589 effects on serum bio-marker TRACP5b (0879).

### DETAILED DESCRIPTION OF THE INVENTION

HDAC inhibitor compounds of particular interest for use in the inventive combination are hydroxamate compounds described by the formula (I):

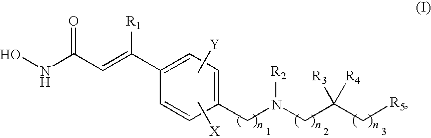

wherein
R₁ is H; halo; or a straight-chain C₁-C₆alkyl, especially methyl, ethyl or n-propyl, which methyl, ethyl and n-propyl substituents are unsubstituted or substituted by one or more substituents described below for alkyl substituents;
R₂ is selected from H; C₁-C₁₀alkyl, preferably C₁-C₆alkyl, e.g., methyl, ethyl or —CH₂CH₂—OH; C₄-C₉cycloalkyl; C₄-C₉heterocycloalkyl; C₄-C₉heterocycloalkylalkyl; cycloalkylalkyl, e.g., cyclopropylmethyl; aryl; heteroaryl; arylalkyl, e.g., benzyl; heteroarylalkyl, e.g., pyridylmethyl; —(CH₂)ₙC(O)R₆; —(CH₂)ₙOC(O)R₆; amino acyl; HON—C(O)—CH═C(R₁)-aryl-alkyl-; and —(CH₂)ₙR₇, R₃ and R₄ are the same or different and independently H, C₁-C₆alkyl, acyl or acylamino, or
R₃ and R₄, together with the carbon to which they are bound, represent C═O, C═S or C═NR₈, or
R₂, together with the nitrogen to which it is bound, and R₃, together with the carbon to which it is bound, can form a C₄-C₉heterocycloalkyl, a heteroaryl, a polyheteroaryl, a non-aromatic polyheterocycle, or a mixed aryl and non-aryl polyheterocycle ring;
R₅ is selected from H; C₁-C₆alkyl; C₄-C₉cycloalkyl; C₄-C₉heterocycloalkyl; acyl; aryl; heteroaryl; arylalkyl, e.g., benzyl; heteroarylalkyl, e.g., pyridylmethyl; aromatic polycycles; non-aromatic polycycles; mixed aryl and non-aryl polycycles; polyheteroaryl; non-aromatic polyheterocycles; and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H; halo; C₁-C₄alkyl, such as CH₃ and CF₃; NO₂; C(O)R₁; OR₉; SR₉; CN; and NR₁₀R₁₁;
R₆ is selected from H; C₁-C₆alkyl; C₄-C₉cycloalkyl; C₄-C₉heterocycloalkyl; cycloalkylalkyl, e.g., cyclopropylmethyl; aryl; heteroaryl; arylalkyl, e.g., benzyl and 2-phenylethenyl; heteroarylalkyl, e.g., pyridylmethyl; OR₁₂; and NR₁₃R₁₄;
R₇ is selected from OR₁₅, SR₁₅, S(O)R₁₆, SO₂R₁₇, NR₁₃R₁₄ and NR₁₂SO₂R₆;
R₈ is selected from H; OR₁₅; NR₁₃R₁₄; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
R₉ is selected from C₁-C₄alkyl, e.g., CH₃ and CF₃; C(O)-alkyl, e.g., C(O)CH₃; and —C(O)CF₃;
R₁₀ and are the same or different and independently selected from H, C₁-C₄alkyl and —C(O)-alkyl;
R₁₂ is selected from H; C₁-C₆alkyl; C₄-C₉cycloalkyl; C₄-C₉heterocycloalkyl; C₄-C₉heterocycloalkylalkyl; aryl; mixed aryl and non-aryl polycycle; heteroaryl; arylalkyl, e.g., benzyl; and heteroarylalkyl, e.g., pyridylmethyl;
R₁₃ and R₁₄ are the same or different and independently selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl; amino acyl, or
R₁₃ and R₁₄, together with the nitrogen to which they are bound, are C₄-C₉heterocycloalkyl, heteroaryl, polyheteroaryl, non-aromatic polyheterocycle or mixed aryl and non-aryl polyheterocycle;
R₁₅ is selected from H, C₄-C₉cycloalkyl, C₄-C₉heterocycloalkyl, aryl, heteroaryl, arylalkyl, heteroarylalkyl and (CH₂)ₘR₁₂;
R₁₆ is selected from C₁-C₆alkyl, C₄-C₉cycloalkyl, C₄-C₉heterocycloalkyl, aryl, heteroaryl, polyheteroaryl, arylalkyl, heteroarylalkyl and (CH₂)ₘZR₁₂;
R₁₇ is selected from C₁-C₆alkyl, C₄-C₉cycloalkyl, C₄-C₉heterocycloalkyl, aryl, aromatic polycycles, heteroaryl, arylalkyl, heteroarylalkyl, polyheteroaryl and NR₁₃R₁₄;
m is an integer selected from 0-6; and
Z is selected from O; NR₁₃; S; and S(O);
   or a pharmaceutically acceptable salt thereof.

As appropriate, “unsubstituted” means that there is no substituent or that the only substituents are hydrogen.

Halo substituents are selected from fluoro, chloro, bromo and iodo, preferably fluoro or chloro.

Alkyl substituents include straight- and branched-C₁-C₆alkyl, unless otherwise noted. Examples of suitable straight- and branched-C₁-C₆alkyl substituents include methyl, ethyl, n-propyl, 2-propyl, n-butyl, sec-butyl, t-butyl and the like. Unless otherwise noted, the alkyl substituents include both unsubstituted alkyl groups and alkyl groups that are substituted by one or more suitable substituents, including unsaturation, i.e., there are one or more double or triple C—C bonds; acyl; cycloalkyl; halo; oxyalkyl; alkylamino; aminoalkyl; acylamino; and OR₁₅, e.g., alkoxy. Preferred substituents for alkyl groups include halo, hydroxy, alkoxy, oxyalkyl, alkylamino and aminoalkyl.

Cycloalkyl substituents include C₃-C₉cycloalkyl groups, such as cyclopropyl, cyclobutyl, cyclopentyl, cyclohexyl and the like, unless otherwise specified. Unless otherwise noted, cycloalkyl substituents include both unsubstituted cycloalkyl groups and cycloalkyl groups that are substituted by one or more suitable substituents, including C₁-C₆alkyl, halo, hydroxy, aminoalkyl, oxyalkyl, alkylamino and OR₁₅, such as alkoxy. Preferred substituents for cycloalkyl groups include halo, hydroxy, alkoxy, oxyalkyl, alkylamino and aminoalkyl.

The above discussion of alkyl and cycloalkyl substituents also applies to the alkyl portions of other substituents, such as, without limitation, alkoxy, alkyl amines, alkyl ketones, arylalkyl, heteroarylalkyl, alkylsulfonyl and alkyl ester substituents and the like.

Heterocycloalkyl substituents include 3- to 9-membered aliphatic rings, such as 4- to 7-membered aliphatic rings, containing from 1-3 heteroatoms selected from nitrogen, sulfur, oxygen. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane and 1,4-oxathiapane. Unless otherwise noted, the rings are unsubstituted or substituted on the carbon atoms by one or more suitable substituents, including C₁-C₆alkyl; C₄-C₉cycloalkyl; aryl; heteroaryl; arylalkyl, e.g., benzyl; heteroarylalkyl, e.g., pyridylmethyl; halo; amino; alkyl amino and OR₁₅, e.g., alkoxy. Unless otherwise noted, nitrogen heteroatoms are unsubstituted or substituted by H, C₁-C₄alkyl; arylalkyl, e.g., benzyl; heteroarylalkyl, e.g., pyridylmethyl; acyl; aminoacyl; alkylsulfonyl; and arylsulfonyl.

Cycloalkylalkyl substituents include compounds of the formula —(CH₂)ₙ₅-cycloalkyl, wherein n5 is a number from 1-6. Suitable alkylcycloalkyl substituents include cyclopentylmethyl, cyclopentylethyl, cyclohexylmethyl and the like. Such substituents are unsubstituted or substituted in the alkyl portion or in the cycloalkyl portion by a suitable substituent, including those listed above for alkyl and cycloalkyl.

Aryl substituents include unsubstituted phenyl and phenyl substituted by one or more suitable substituents including C₁-C₆alkyl; cycloalkylalkyl, e.g., cyclopropylmethyl; O(CO)alkyl; oxyalkyl; halo; nitro; amino; alkylamino; aminoalkyl; alkyl ketones; nitrile; carboxyalkyl; alkylsulfonyl; aminosulfonyl; arylsulfonyl and OR₁₅, such as alkoxy.

Preferred substituents include including C₁-C₆alkyl; cycloalkyl, e.g., cyclopropylmethyl; alkoxy; oxyalkyl; halo; nitro; amino; alkylamino; aminoalkyl; alkyl ketones; nitrile; carboxyalkyl; alkylsulfonyl; arylsulfonyl and aminosulfonyl. Examples of suitable aryl groups include C₁-C₄alkylphenyl, C₁-C₄alkoxyphenyl, trifluoromethylphenyl, methoxyphenyl, hydroxyethylphenyl, dimethylaminophenyl, aminopropylphenyl, carbethoxyphenyl, methanesulfonylphenyl and tolylsulfonylphenyl.

Aromatic polycycles include naphthyl, and naphthyl substituted by one or more suitable substituents including C₁-C₆alkyl; alkylcycloalkyl, e.g., cyclopropylmethyl; oxyalkyl; halo; nitro; amino; alkylamino; aminoalkyl; alkyl ketones; nitrile; carboxyalkyl; alkylsulfonyl; arylsulfonyl; aminosulfonyl and OR₁₅, such as alkoxy.

Heteroaryl substituents include compounds with a 5- to 7-membered aromatic ring containing one or more heteroatoms, e.g., from 1-4 heteroatoms, selected from N, O and S. Typical heteroaryl substituents include furyl, thienyl, pyrrole, pyrazole, triazole, thiazole, oxazole, pyridine, pyrimidine, isoxazolyl, pyrazine and the like. Unless otherwise noted, heteroaryl substituents are unsubstituted or substituted on a carbon atom by one or more suitable substituents, including alkyl, the alkyl substituents identified above, and another heteroaryl substituent. Nitrogen atoms are unsubstituted or substituted, e.g., by R₁₃; especially useful N substituents include H, C₁-C₄alkyl, acyl, aminoacyl and sulfonyl.

Arylalkyl substituents include groups of the formula —(CH₂)ₙ₅-aryl, —(CH₂)ₙ₅₋₁—(CH-aryl)-(CH₂)ₙ₅-aryl or —(CH₂)ₙ₅₋₁CH(aryl)(aryl), wherein aryl and n5 are defined above. Such arylalkyl substituents include benzyl, 2-phenylethyl, 1-phenylethyl, tolyl-3-propyl, 2-phenylpropyl, diphenylmethyl, 2-diphenylethyl, 5,5-dimethyl-3-phenylpentyl and the like. Arylalkyl substituents are unsubstituted or substituted in the alkyl moiety or the aryl moiety or both as described above for alkyl and aryl substituents.

Heteroarylalkyl substituents include groups of the formula —(CH₂)ₙ₅-heteroaryl, wherein heteroaryl and n5 are defined above and the bridging group is linked to a carbon or a nitrogen of the heteroaryl portion, such as 2-, 3- or 4-pyridylmethyl, imidazolylmethyl, quinolylethyl and pyrrolylbutyl. Heteroaryl substituents are unsubstituted or substituted as discussed above for heteroaryl and alkyl substituents.

Amino acyl substituents include groups of the formula —C(O)—(CH₂)ₙ—C(H)(NR₁₃R₁₄)—(CH₂)ₙ—R₅, wherein n, R₁₃, R₁₄ and R₅ are described above. Suitable aminoacyl substituents include natural and non-natural amino acids, such as glycinyl, D-tryptophanyl, L-lysinyl, D- or L-homoserinyl, 4-aminobutryic acyl and ±-3-amin-4-hexenoyl.

Non-aromatic polycycle substituents include bicyclic and tricyclic fused ring systems where each ring can be 4- to 9-membered and each ring can contain zero, one or more double and/or triple bonds. Suitable examples of non-aromatic polycycles include decalin, octahydroindene, perhydrobenzocycloheptene and perhydrobenzo-(t)-azulene. Such substituents are unsubstituted or substituted as described above for cycloalkyl groups.

Mixed aryl and non-aryl polycycle substituents include bicyclic and tricyclic fused ring systems where each ring can be 4- to 9-membered and at least one ring is aromatic. Suitable examples of mixed aryl and non-aryl polycycles include methylenedioxyphenyl, bis-methylenedioxyphenyl, 1,2,3,4-tetrahydronaphthalene, dibenzosuberane, dihdydroanthracene and 9H-fluorene. Such substituents are unsubstituted or substituted by nitro or as described above for cycloalkyl groups.

Polyheteroaryl substituents include bicyclic and tricyclic fused ring systems where each ring can independently be 5- or 6-membered and contain one or more heteroatom, e.g., 1, 2, 3 or 4 heteroatoms, chosen from O, N or S such that the fused ring system is aromatic. Suitable examples of polyheteroaryl ring systems include quinoline, isoquinoline, pyridopyrazine, pyrrolopyridine, furopyridine, indole, benzofuran, benzothiofuran, benzindole, benzoxazole, pyrroloquinoline and the like. Unless otherwise noted, polyheteroaryl substituents are unsubstituted or substituted on a carbon atom by one or more suitable substituents, including alkyl, the alkyl substituents identified above and a substituent of the formula —O—(CH₂CH═CH(CH₃)(CH₂))₁₋₃H. Nitrogen atoms are unsubstituted or substituted, e.g., by R₁₃, especially useful N substituents include H, C₁-C₄alkyl, acyl, aminoacyl and sulfonyl.

Non-aromatic polyheterocyclic substituents include bicyclic and tricyclic fused ring systems where each ring can be 4- to 9-membered, contain one or more heteroatom, e.g., 1, 2, 3 or 4 heteroatoms, chosen from O, N or S and contain zero or one or more C—C double or triple bonds. Suitable examples of non-aromatic polyheterocycles include hexitol, cis-perhydro-cyclohepta[b]pyridinyl, decahydro-benzo[f][1,4]oxazepinyl, 2,8-dioxabicyclo[3.3.0]octane, hexahydro-thieno[3,2-b]thiophene, perhydropyrrolo[3,2-b]pyrrole, perhydronaphthyridine, perhydro-1H-dicyclopenta[b,e]pyran. Unless otherwise noted, non-aromatic polyheterocyclic substituents are unsubstituted or substituted on a carbon atom by one or more substituents, including alkyl and the alkyl substituents identified above. Nitrogen atoms are unsubstituted or substituted, e.g., by R₁₃, especially useful. N substituents include H, C₁-C₄alkyl, acyl, aminoacyl and sulfonyl.

Mixed aryl and non-aryl polyheterocycles substituents include bicyclic and tricyclic fused ring systems where each ring can be 4- to 9-membered, contain one or more heteroatom chosen from O, N or S, and at least one of the rings must be aromatic. Suitable examples of mixed aryl and non-aryl polyheterocycles include 2,3-dihydroindole, 1,2,3,4-tetrahydroquinoline, 5,11-dihydro-10H-dibenz[b,e][1,4]diazepine, 5H-dibenzo[b,e][1,4]diazepine, 1,2-dihydropyrrolo[3,4-b][1,5]benzodiazepine, 1,5-dihydro-pyrido[2,3-b][1,4]diazepin-4-one, 1,2,3,4,6,11-hexahydro-benzo[b]pyrido[2,3-e][1,4]diazepin-5-one. Unless otherwise noted, mixed aryl and non-aryl polyheterocyclic substituents are unsubstituted or substituted on a carbon atom by one or more suitable substituents including —N—OH, ═N—OH, alkyl and the alkyl substituents identified above. Nitrogen atoms are unsubstituted or substituted, e.g., by R₁₃; especially useful N substituents include H, C₁-C₄alkyl, acyl, aminoacyl and sulfonyl.

Amino substituents include primary, secondary and tertiary amines and in salt form, quaternary amines. Examples of amino substituents include mono- and di-alkylamino, mono- and di-aryl amino, mono- and di-arylalkyl amino, aryl-arylalkylamino, alkyl-arylamino, alkyl-arylalkylamino and the like.

Sulfonyl substituents include alkylsulfonyl and arylsulfonyl, e.g., methane sulfonyl, benzene sulfonyl, tosyl and the like.

Acyl substituents include groups of formula —C(O)—W, —OC(O)—W, —C(O)—O—W or —C(O)NR₁₃R₁₄, where W is R₁₆, H or cycloalkylalkyl.

Acylamino substituents include substituents of the formula —N(R₁₂)C(O)—W, —N(R₁₂)C(O)—O—W and —N(R₁₂)C(O)—NHOH and R₁₂ and W are defined above.

The R₂ substituent HON—C(O)—CH═C(R₁)-aryl-alkyl- is a group of the formula:

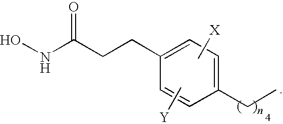

Preferences for each of the substituents include the following:
R₁ is H, halo or a straight-chain C₁-C₄alkyl;
R₂ is selected from H, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇,
R₃ and R₄ are the same or different and independently selected from H and C₁-C₆alkyl, or
R₃ and R₄, together with the carbon to which they are bound, represent C═O, C═S or C═NR₈;
R₅ is selected from H, C₁-C₆alkyl, C₄-C₉cycloalkyl, C₄-C₉heterocycloalkyl, aryl, heteroaryl, arylalkyl, heteroarylalkyl, a aromatic polycycle, a non-aromatic polycycle, a mixed aryl and non-aryl polycycle, polyheteroaryl, a non-aromatic polyheterocycle, and a mixed aryl and non-aryl polyheterocycle;
n, n₁, n₂ and n₃ are the same or different and independently selected from 0-6, when n₁ is 1-6, each carbon atom is unsubstituted or independently substituted with R₃ and/or R₄;
X and Y are the same or different and independently selected from H, halo, C₁-C₄alkyl, CF₃, NO₂, C(O)R₁, OR₉, SR₉, CN and NR₁₀R₁₁;
R₆ is selected from H, C₁-C₆alkyl, C₄-C₉cycloalkyl, C₄-C₉heterocycloalkyl, alkylcycloalkyl, aryl, heteroaryl, arylalkyl, heteroarylalkyl, OR₁₂ and NR₁₃R₁₄;
R₇ is selected from OR₁₅, SR₁₅, S(O)R₁₆, SO₂R₁₇, NR₁₃R₁₄ and NR₁₂SO₂R₆;
R₈ is selected from H, OR₁₅, NR₁₃R₁₄, C₁-C₆alkyl, C₄-C₉cycloalkyl, C₄-C₉heterocycloalkyl, aryl, heteroaryl, arylalkyl and heteroarylalkyl;
R₉ is selected from C₁-C₄alkyl and C(O)-alkyl;
R₁₀ and R₁₁ are the same or different and independently selected from H, C₁-C₄alkyl and —C(O)-alkyl;
R₁₂ is selected from H, C₄-C₉cycloalkyl, C₄-C₉heterocycloalkyl, aryl, heteroaryl, arylalkyl and heteroarylalkyl;
R₁₃ and R₁₄ are the same or different and independently selected from H, C₁-C₆alkyl, C₄-C₉cycloalkyl, C₄-C₉heterocycloalkyl, aryl, heteroaryl, arylalkyl, heteroarylalkyl and amino acyl;
R₁₅ is selected from H, C₁-C₆alkyl, C₄-C₉cycloalkyl, C₄-C₉heterocycloalkyl, aryl, heteroaryl, arylalkyl, heteroarylalkyl and (CH₂)ₘZR₁₂,
R₁₆ is selected from C₁-C₆alkyl, C₄-C₉cycloalkyl, C₄-C₉heterocycloalkyl, aryl, heteroaryl, arylalkyl, heteroarylalkyl and (CH₂)ₘZR₁₂;
R₁₇ is selected from C₁-C₆alkyl, C₄-C₉cycloalkyl, C₄-C₉heterocycloalkyl, aryl, heteroaryl, arylalkyl, heteroarylalkyl and NR₁₃R₁₄;
m is an integer selected from 0-6; and
Z is selected from O, NR₁₃, S and S(O);
   or a pharmaceutically acceptable salt thereof.

Useful compounds of the formula (I), include those wherein each of R₁, X, Y, R₃ and R₄ is H, including those wherein one of n₂ and n₃ is 0 and the other is 1, especially those wherein R₂ is H or —CH₂—CH₂—OH.

One suitable genus of hydroxamate compounds are those of formula (Ia):

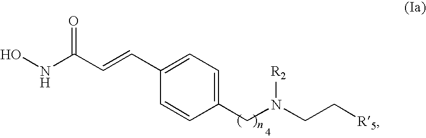

wherein
n₄ is 0-3;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)₆R₇; and
R₅′ is heteroaryl; heteroarylalkyl, e.g., pyridylmethyl; aromatic polycycles; non-aromatic polycycles; mixed aryl and non-aryl polycycles; polyheteroaryl or mixed aryl; and non-aryl polyheterocycles;
   or a pharmaceutically acceptable salt thereof.

Another suitable genus of hydroxamate compounds are those of formula (Ia):

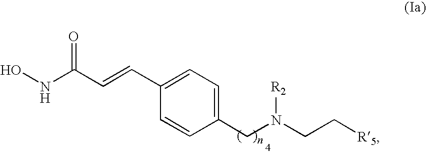

wherein
n₄ is 0-3;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
R₅′ is aryl; arylalkyl; aromatic polycycles; non-aromatic polycycles and mixed aryl; and non-aryl polycycles, especially aryl, such as p-fluorophenyl, p-chlorophenyl, p-O—C₁-C₄alkylphenyl, such as p-methoxyphenyl, and p-C₁-C₄alkylphenyl; and arylalkyl, such as benzyl, ortho-, meta- or para-fluorobenzyl, ortho-, meta- or para-chlorobenzyl, ortho-, meta- or para-mono, di- or tri-O—C₁-C₄alkylbenzyl, such as ortho-, meta- or para-methoxybenzyl, m,p-diethoxybenzyl, o,m,p-triimethoxybenzyl and ortho-, meta- or para-mono, di- or tri-C₁-C₄alkylphenyl, such as p-methyl, m,m-diethylphenyl;
   or a pharmaceutically acceptable salt thereof.

Another interesting genus is the compounds of formula (Ib):

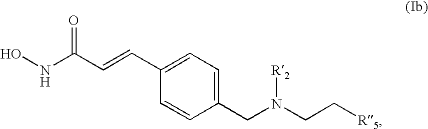

wherein
R₂′ is selected from H; C₁-C₆alkyl; C₄-C₆cycloalkyl; cycloalkylalkyl, e.g., cyclopropylmethyl; (CH₂)₂₋₄OR₂₁, where R₂₁ is H, methyl, ethyl, propyl and i-propyl; and
R₅″ is unsubstituted 1H-indol-3-yl, benzofuran-3-yl or quinolin-3-yl, or substituted 1H-indol-3-yl, such as 5-fluoro-1H-indol-3-yl or 5-methoxy-1H-indol-3-yl, benzofuran-3-yl or quinolin-3-yl;
   or a pharmaceutically acceptable salt thereof.

Another interesting genus of hydroxamate compounds are the compounds of formula (Ic):

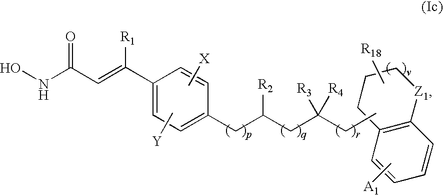

wherein
the ring containing Z₁ is aromatic or non-aromatic, which non-aromatic rings are saturated or unsaturated;
Z₁ is O, S or N—R₂₀;

R₁₈ is H; halo; C₁-C₆alkyl (methyl, ethyl, t-butyl); C₃-C₇cycloalkyl; aryl, e.g., unsubstituted phenyl or phenyl substituted by 4-OCH₃ or 4-CF₃; or heteroaryl, such as 2-furanyl, 2-thiophenyl or 2-, 3- or 4-pyridyl;
R₂₀ is H; C₁-C₆alkyl; C₁-C₆alkyl-C₃-C₉cycloalkyl, e.g., cyclopropylmethyl; aryl; heteroaryl; arylalkyl, e.g., benzyl; heteroarylalkyl, e.g., pyridylmethyl; acyl, e.g., acetyl, propionyl and benzoyl; or sulfonyl, methanesulfonyl, ethanesulfonyl, benzenesulfonyl and toluenesulfonyl;
A₁ is 1, 2 or 3 substituents which are independently H; C₁-C₆alkyl; —OR₁₉; halo; alkylamino; aminoalkyl; halo; or heteroarylalkyl, e.g., pyridylmethyl;
R₁₉ is selected from H; C₁-C₆alkyl; C₄₋₉cycloalkyl; C₄-C₉heterocycloalkyl; aryl; heteroaryl; arylalkyl, e.g., benzyl; heteroarylalkyl, e.g., pyridylmethyl and —(CH₂CH═CH(CH₃)(CH₂))₁₋₃H,
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
v is 0, 1 or 2;
p is 0-3; and
q is 1-5 and r is 0, or
q is 0 and r is 1-5;
   or a pharmaceutically acceptable salt thereof. The other variable substituents are as defined above.

Especially useful compounds of formula (Ic), are those wherein R₂ is H, or —(CH₂)ₚCH₂OH, wherein p is 1-3, especially those wherein R₁ is H, such as those wherein R₁ is H and X and Y are each H, and wherein q is 1-3 and r is 0 or wherein q is 0 and r is 1-3, especially those wherein Z₁ is N—R₂₀. Among these compounds R₂ is preferably H or —CH₂—CH₂—OH and the sum of q and r is preferably 1.

Another interesting genus of hydroxamate compounds are the compounds of formula (Id):

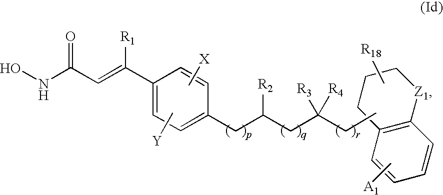

wherein
Z₁ is O, S or N—R₂₀;
R₁₈ is H; halo; C₁-C₆alkyl (methyl, ethyl, t-butyl); C₃-C₂cycloalkyl; aryl, e.g., unsubstituted phenyl or phenyl substituted by 4-OCH₃ or 4-CF₃; or heteroaryl;
R₂₀ is H; C₁-C₆alkyl, C₁-C₆alkyl-C₃-C₉cycloalkyl, e.g., cyclopropylmethyl; aryl; heteroaryl; arylalkyl, e.g., benzyl; heteroarylalkyl, e.g., pyridylmethyl; acyl, e.g., acetyl, propionyl and benzoyl; or sulfonyl, e.g., methanesulfonyl, ethanesulfonyl, benzenesulfonyl, toluenesulfonyl);
A₁ is 1, 2 or 3 substituents which are independently H, C₁-C₆alkyl, —OR₁₉ or halo;
R_{1B} is selected from H; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
p is 0-3; and
q is 1-5 and r is 0, or
q is 0 and r is 1-5;
   or a pharmaceutically acceptable salt thereof. The other variable substituents are as defined above.

Especially useful compounds of formula (Id), are those wherein R₂ is H or —(CH₂)ₚCH₂OH, wherein p is 1-3, especially those wherein R₁ is H, such as those wherein R₁ is H and X and Y are each H, and wherein q is 1-3 and r is 0 or wherein q is 0 and r is 1-3. Among these compounds R₂ is preferably H, or —CH₂—CH₂—OH and the sum of q and r is preferably 1.

The present invention further relates to compounds of the formula (Ie):

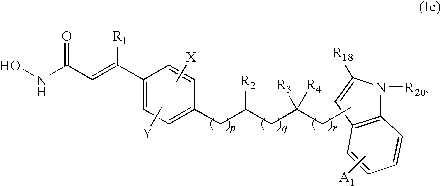

or a pharmaceutically acceptable salt thereof. The variable substituents are as defined above.

Especially useful compounds of formula (Ie), are those wherein R₁₆ is H, fluoro, chloro, bromo, a C₁-C₄alkyl group, a substituted C₁-C₄alkyl group, a C₃-C₇cycloalkyl group, unsubstituted phenyl, phenyl substituted in the para position, or a heteroaryl, e.g., pyridyl, ring.

Another group of useful compounds of formula (Ie), are those wherein R₂ is H or —(CH₂)ₚCH₂OH, wherein p is 1-3, especially those wherein R₁ is H, such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1. Among these compounds p is preferably 1 and R₃ and R₄ are preferably H.

Another group of useful compounds of formula (Ie), are those wherein R₁₈ is H, methyl, ethyl, t-butyl, trifluoromethyl, cyclohexyl, phenyl, 4-methoxyphenyl, 4-trifluoromethylphenyl, 2-furanyl, 2-thiophenyl, or 2-, 3- or 4-pyridyl wherein the 2-furanyl, 2-thiophenyl and 2-, 3- or 4-pyridyl substituents are unsubstituted or substituted as described above for heteroaryl rings; R₂ is H or —(CH₂)ₚCH₂OH, wherein p is 1-3; especially those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

Those compounds of formula (Ie), wherein R₂₀ is H or C₁-C₆alkyl, especially H, are important members of each of the subgenuses of compounds of formula (Ie) described above.

N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide or a pharmaceutically acceptable salt thereof, are important compounds of formula (Ie).

The present invention further to the compounds of the formula (If):

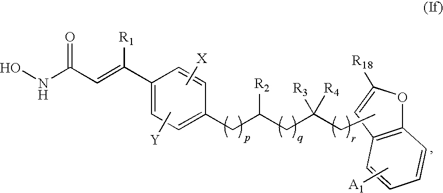

or a pharmaceutically acceptable salt thereof. The variable substituents are as defined above.

Useful compounds of formula (If), are include those wherein R₂ is H or —(CH₂)ₚCH₂OH, wherein p is 1-3, especially those wherein R₁ is H, such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

N-hydroxy-3-[4-[[[2-(benzofur-3-yl)-ethyl]-amino]methyl]phenyl]-2E-2-propenamide or a pharmaceutically acceptable salt thereof, is an important compound of formula (If).

The compounds described above are often used in the form of a pharmaceutically acceptable salt. Pharmaceutically acceptable salts include, when appropriate, pharmaceutically acceptable base addition salts and acid addition salts, e.g., metal salts, such as alkali and alkaline earth metal salts, ammonium salts, organic amine addition salts and amino acid addition salts and sulfonate salts. Acid addition salts include inorganic acid addition salts, such as hydrochloride, sulfate and phosphate; and organic acid addition salts, such as alkyl sulfonate, arylsulfonate, acetate, maleate, fumarate, tartrate, citrate and lactate. Examples of metal salts are alkali metal salts, such as lithium salt, sodium salt and potassium salt; alkaline earth metal salts, such as magnesium salt and calcium salt, aluminum salt and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

Additional HDAI compounds within the scope of formula (I), and their synthesis, are disclosed in WO 02/22577. Two preferred compounds within the scope of WO 02/22577 are:

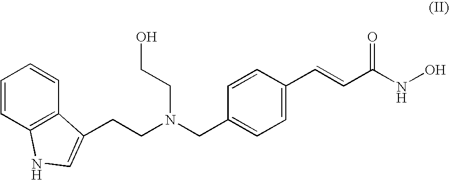

N-hydroxy-3-[4-[(2-hydroxyethyl){2-(1H-indol-3-yl)ethyl]-amino]methyl]phenyl]-2E-2-propenamide, or a pharmaceutically acceptable salt thereof; and

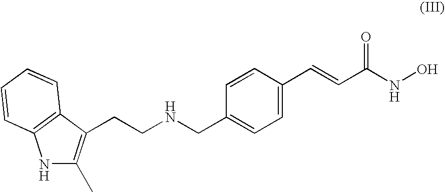

N-hydroxy-3-[4-[[[2-(2-methyl-1H-indol-3-yl)-ethyl]-amino]methyl]phenyl]-2E-2-propenamide, or a pharmaceutically acceptable salt thereof.

An HDAC inhibitor as used for the present invention displays in the assay described above preferably an IC₅₀ value between 50 and 2500 nM, more preferably between 250 and 2000 nM, and most preferably between 500 and 1250 nM.

The term “treatment”, as used herein, comprises the treatment of patients having bone destruction caused by cancer, inflammatory diseases and osteoporsis.

In one embodiment, N-hydroxy-3-[4-[[[2-(2-methyl-1H-indol-3-yl)-ethyl]-amino]methyl]phenyl]-2E-2-propenamide or a pharmaceutically acceptable salt thereof is used to treat bone destruction associated with multiple myeloma. Hence, the invention relates to the use of N-hydroxy-3-[4-[[[2-(2-methyl-1H-indol-3-yl)-ethyl]-amino]methyl]phenyl]-2E-2-propenamide or a pharmaceutically acceptable salt thereof for the preparation of a medicament for the treatment of bone destruction associated with multiple myeloma. The invention also relates to the use of N-hydroxy-3-[4-[[[2-(2-methyl-1H-indol-3-yl)-ethyl]-amino]methyl]phenyl]-2E-2-propenamide or a pharmaceutically acceptable salt thereof in the treatment of bone destruction associated with multiple myeloma. The invention relates to a method of treating warm-blooded animals including mammals, especially humans, suffering from bone destruction associated with multiple myeloma by administering to a said animal in need of such treatment a dose effective against said disease of N-hydroxy-3-[4-[[[2-(2-methyl-1H-indol-3-yl)-ethyl]-amino]methyl]phenyl]-2E-2-propenamide or a pharmaceutically acceptable salt thereof or a pharmaceutically acceptable salt thereof.

In another embodiment, N-hydroxy-3-[4-[[[2-(2-methyl-1H-indol-3-yl)-ethyl]-amino]methyl]phenyl]-2E-2-propenamide or a pharmaceutically acceptable salt thereof is used to treat bone destruction associated with breast cancer. Hence, the invention relates to the use of N-hydroxy-3-[4-[[[2-(2-methyl-1H-indol-3-yl)-ethyl]-amino]methyl]phenyl]-2E-2-propenamide or a pharmaceutically acceptable salt thereof for the preparation of a medicament for the treatment of bone destruction associated with breast cancer. The invention also relates to the use of N-hydroxy-3-[4-[[[2-(2-methyl-1H-indol-3-yl)-ethyl]-amino]methyl]phenyl]-2E-2-propenamide or a pharmaceutically acceptable salt thereof in the treatment of bone destruction associated with breast cancer. The invention relates to a method of treating warm-blooded animals including mammals, especially humans, suffering from bone destruction associated with breast cancer by administering to a said animal in need of such treatment a dose effective against said disease of N-hydroxy-3-[4-[[[2-(2-methyl-1H-indol-3-yl)-ethyl]-amino]methyl]phenyl]-2E-2-propenamide or a pharmaceutically acceptable salt thereof or a pharmaceutically acceptable salt thereof.

In another embodiment, N-hydroxy-3-[4-[[[2-(2-methyl-1H-indol-3-yl)-ethyl]-amino]methyl]phenyl]-2E-2-propenamide or a pharmaceutically acceptable salt thereof is used to treat bone destruction associated with prostate cancer. Hence, the invention relates to the use of N-hydroxy-3-[4-[[[(2-(2-methyl-1H-indol-3-yl)-ethyl]-amino]methyl]phenyl]-2E-2-propenamide or a pharmaceutically acceptable salt thereof for the preparation of a medicament for the treatment of bone destruction associated with prostate cancer. The invention also relates to the use of N-hydroxy-3-[4-[[[2-(2-methyl-1H-indol-3-yl)-ethyl]-amino]methyl]phenyl]-2E-2-propenamide or a pharmaceutically acceptable salt thereof in the treatment of bone destruction associated with prostate cancer. The invention relates to a method of treating warm-blooded animals including mammals, especially humans, suffering from bone destruction associated with prostate cancer by administering to a said animal in need of such treatment a dose effective against said disease of N-hydroxy-3-[4-[[[2-(2-methyl-1H-indol-3-yl)-ethyl]-amino]methyl]phenyl]-2E-2-propenamide or a pharmaceutically acceptable salt thereof.

In another embodiment, N-hydroxy-3-[4-[[[2-(2-methyl-1H-indol-3-yl)-ethyl]-amino]methyl]phenyl]-2E-2-propenamide or a pharmaceutically acceptable salt thereof is used to treat bone destruction associated with inflammatory diseases. Hence, the invention relates to the use of N-hydroxy-3-[4-[[[(2-(2-methyl-1H-indol-3-yl)-ethyl]-amino]methyl]phenyl]-2E-2-propenamide or a pharmaceutically acceptable salt thereof for the preparation of a medicament for the treatment of bone destruction associated with inflammatory diseases. The invention also relates to the use of N-hydroxy-3-[4-[[[2-(2-methyl-1H-indol-3-yl)-ethyl]-amino]methyl]phenyl]-2E-2-propenamide or a pharmaceutically acceptable salt thereof in the treatment of bone destruction associated with inflammatory diseases. The invention relates to a method of treating warm-blooded animals including mammals, especially humans, suffering from bone destruction associated with inflammatory diseases by administering to a said animal in need of such treatment a dose effective against said disease of N-hydroxy-3-[4-[[[2-(2-methyl-1H-indol-3-yl)-ethyl]-amino]methyl]phenyl]-2E-2-propenamide or a pharmaceutically acceptable salt thereof.

In another embodiment, it was found that N-hydroxy-3-[4-[[[2-(2-methyl-1H-indol-3-yl)-ethyl]-amino]methyl]phenyl]-2E-2-propenamide or a pharmaceutically acceptable salt thereof, treat bone destruction associated with osteoporosis. Hence, the invention relates to the use of N-hydroxy-3-[4-[[[2-(2-methyl-1H-indol-3-yl)-ethyl]-amino]methyl]phenyl]-2E-2-propenamide or a pharmaceutically acceptable salt thereof for the preparation of a medicament for the treatment of bone destruction associated with osteoporosis. The invention also relates to the use of N-hydroxy-3-[4-[[[2-(2-methyl-1H-indol-3-yl)-ethyl]-amino]methyl]phenyl]-2E-2-propenamide or a pharmaceutically acceptable salt thereof in the treatment of bone destruction associated with osteoporosis. The invention relates to a method of treating warm-blooded animals including mammals, especially humans, suffering from bone destruction associated with osteoporosis by administering to a said animal in need of such treatment a dose effective against said disease of N-hydroxy-3-[4-[[[2-(2-methyl-1H-indol-3-yl)-ethyl]-amino]methyl]phenyl]-2E-2-propenamide or a pharmaceutically acceptable salt thereof.

The person skilled in the pertinent art is fully enabled to select relevant test models to prove the beneficial effects mentioned herein. The pharmacological activity of such a compound may, e.g., be demonstrated by means of the Examples described below, by in vitro tests and in vivo tests or in suitable clinical studies. The efficacy of the treatment is determined in these studies, e.g., by evaluation of the tumor sizes every 4 weeks, with the control achieved on placebo.

The effective dosage of the HDAC inhibitor may vary depending on the particular compound or pharmaceutical composition employed, on the mode of administration, the type of the disease being treated or its severity. The dosage regimen is selected in accordance with a variety of further factors including the renal and hepatic function of the patient. A physician, clinician or veterinarian of ordinary skill can readily determine and prescribe the effective amount of compounds required to prevent, counter or arrest the progress of the condition.

### Example 1

Female SCID-beige mice were injected with MM1S cells (2×10⁶) intravenously (iv) into the tail vein on Day 0. Treatment was initiated on Day 10 when the average tumor burden, as determined by bioluminescence, reached approximately 8.0×10⁵-1.0×10⁶ photons per second. All treatment groups consisted of 8 animals. LBH589 was dosed at 15 mg/kg ip qdX5 for 3 weeks in the first study. In the second study, LBH589 was dosed at 10 mg/kg ip qdX5 for 6 weeks and 20 mg/kg ip qdX5 for 5 weeks. Tumor burden and body weights were recorded once a week during the active dosing period. In vivo micro-computed tomography (microCT) images were acquired in live animals on day 32 and 33 (Study #1) or Day 34 and 35 (Study #2). In the second study, animals were individually monitored until achievement of clinical endpoint.

In Vivo microCT or μCT Analysis

Animals were anesthetized with 2% isoflurane mixed with oxygen (2 L/min.) and then placed in a mouse holder (custom made, Peter Ingold, NIBR Basel) specifically designed to align both tibiae and mounted into an in vivo high resolution microCT scanner (VivaCT40, Scanco, Switzerland). To insure correct positioning of the mouse, a scout view of bilateral tibia bones and knee joints was taken and the region of interest (ROI, 2.23 mm in length) was positioned to start at the growth plate extending distally over the area of the trabecular bone (FIG. 3). The scanner was set to a nominal isotropic voxel size of 21 μm, referred to as medium/standard resolution. The X-ray tube was operated at 55 kVp and 145 mA with a focal spot size of 5 μm. Five hundred projection images were acquired per scan with an integration time of 180 ms. Tomographic images were reconstructed on a VMS cluster (HP Alpha, HP, Palo Alto, USA) in 1024×1024 pixel matrices using a conebeam back projection procedure resulting in 315 axial slices.

For determination of trabecular and cortical bone features, a 2.23 mm region of interest was placed to start at the growth plate extending distally. 106 axial slices were obtained using a μ-CT VivaCT40 Scanner (SCANCO, Switzerland) with 55 kv, 145 mA, 180 ms integration time and 21 μm resolution. Trabecular bone density (BV/TV) was measured in a 0.735 mm region of a tibia (9 slices proximal and 25 slices distal from the tibial tuberosity) using SCANCO software (SCANCO, Switzerland) with a threshold of 275 is used to define calcified bone volume (BV). Cortical bone density (BV/TV) was measured in a 1.5 mm region of a tibia (15 slices proximal and 55 slices distal from the tibial tuberosity) using SCANCO software (SCANCO Switzerland) with a threshold of 275. Three-dimensional analysis was performed on the determined regions utilizing the SCANCO operational software. All treatment groups were scanned over the course of two days, with equal numbers of animals from each treatment group scanned each day.

### Serum Bio-Marker Analysis

A serum marker of bone metabolism, TRACP5B, was assessed for mouse serum changes. The MouseTRAP™ Assay kit is an ELISA assay (Cat#SB-TR103, IDS Fountain Hills, Ariz.). Briefly, polyclonal mouse TRACP5B antibodies are incubated in 96 well plates coated with anti-rabbit IgG. This ELISA kit is specific for mouse TRACP5B only. This assay has a reported sensitivity of 0.1 U/L.

### LBH589 Effects on Tumor Burden

Following tail vein injection, MM1S cells proliferated and tumor burden increased over 1,400 to 2.300-fold as determined by bioluminescent readout. MM1S cells localized to bone resulting in multifocal bone lesions in the vertebrae, ribs, skull, pelvis and long bones consistent with human clinical presentation.

The mean relative change in tumor burden expressed as luciferase flux (photons per second) are shown in Tables 1 and 2:

TABLE 1
Response Summary for Study #0879 31 Days After Implantation

| | | | TUMORS | | | | |
|---|---|---|---|---|---|---|---|
| | | | Delta Mean | | | | |
| DRUGS | | | Tumor Burden | | | ANIMALS | |
| | Regimen | Dose | (photons/sec) | % | % | Delta % | Dead/ |
| Compound | & Route | (mg/kg) | (mean ± SEM) | T/C | Regression | Body Wt. | Total |
| Vehicle | D5W, IP, qdx5 | N/A | 1.3E+9 ± 261.8E+6 | NE | NE | −3.8 ± 1.47 | 0/8 |
| LBH589 | IP, qdx5 | 15 | 292.4E+6 ± 92.6E+6 | 22 | None | −10.5 ± 0.99 | 1/8 |

Treatments were started on Day 10 post-iv tail implantation (2.0 million cells/animal). LBH589 was administered ip, at 15 mg/kg, 5 times per week for 3 weeks. Vehicle control (D5W) was administered ip times per week, for 3 weeks. Initial group size: 8 animals. Final efficacy data and body weight change were calculated 72 hours post-last dose.

TABLE 2
Response Summary for Study #0942 35 Days After Implantation

| | | | TUMORS | | | | |
|---|---|---|---|---|---|---|---|
| | | | Delta Mean | | | | |
| DRUGS | | | Tumor Burden | | | ANIMALS | |
| | Regimen | Dose | (photons/sec) | % | % | Delta % | Dead/ |
| Compound | & Route | (mg/kg) | (mean ± SEM) | T/C | Regression | Body Wt. | Total |
| Vehicle | D5W, IP, qdx5 | N/A | 1.1E+9 ± 171.4E+6 | NE | NE | 2 ± 1.81 | 1/8 |
| LBH589 | IP, qdx5 | 10 | 348.5E+6 ± 49.6E+6 | 31 | None | −11.3 ± 0.89 | 0/8 |
| LBH589 | IP, qdx5 | 20 | 107.7E+6 ± 42.6E+6 | 9 | None | −13.1 ± 1.22 | 0/8 |

Treatments were started on Day 10 post-iv tail implantation (2.0 million cells/animal). LBH589 was administered ip, at 10 or 20 mg/kg, 5 times per week for 4 weeks. Vehicle control (D5W) was administered ip times per week, for 3 weeks. Initial group size: 8 animals.

Statistical analyses of final tumor burden are presented in Tables 3 and Table 4.

TABLE 3
0879 Statistics of Day 31 Delta Tumor Burden

| Treatments (#0879) | Vehicle | LBH589 (15) |
|---|---|---|
| Vehicle | X | S |
| LBH589 (15) | | x |

Students T-Test
S = P < 0.01

TABLE 4
0942 Statistics of Day 35 Delta Tumor Burden

| Treatments (#0879) | Vehicle | LBH589 (10) | LBH589 (20) |
|---|---|---|---|
| Vehicle | X | S | S |
| LBH589 (10) | | X | NS |
| LBH589 (20) | | | X |

ANOVA + Dunnett's Method Post-hoc test
S = P < 0.05
NS = Not Significant

As illustrated in FIG. 1, FIG. 2, Table 1 and Table 2, tumor burden increased greater than 1.400-fold over the 4-5 week post-implantation period in the D5W-treated controls groups in Study #0879 and over 2.300-fold over the 5-week post-implantation period in the D5W-treated control groups in Study #0942.

With respect to FIG. 2, treatments were started on Day 11 post-iv tail implantation (2.0 million cells/animal). NVP-LBH589-CU was administered ip, at 15 mg/kg (A), or 10 and 20 mg/kg (B), 5 times a week (qd×5/wk) for 4 weeks. Vehicle control (D5W) was administered ip 5 times a week (qd×5/wk), for 4 weeks. Bortezomib was administered iv, at 0.2 mg/kg, once per week (qwk) or 1 mg/kg, twice per week in Study #0879 for 4 weeks (biwk) (B). Initial group size: 8 animals. Final efficacy data are shown in the A panel for Study #0879 and B panel for Study #0942. Body weight changes were calculated on 24 hours post-last dose for each study (right panels).

LBH589 treatment at 15 mg/kg qd×5 alone resulted in a reduction in tumor burden by ˜78% on Day 31 in Study #0879. LBH589 treatment at 10 mg/kg qdX5 or 20 mg/kg qdX5 alone resulted in a reduction in tumor burden by ˜79% and ˜91%, respectively on Day 35 in Study #0942. The reduction in tumor burden by LBH589 was statistically significant in both studies.

### LBH589 Effects on Time to Endpoint

The ability of LBH589 to extend the time to clinical endpoint was evaluated in Study #0942. Each animal was monitored daily for progression of signs of disease progression, including mobility and general health. Animals were scored on a clinical scale from 0-4. Endpoint was achieved when animals achieved a clinical score of 3. The effects of LBH589 on increasing time to endpoint are shown in FIG. 3 and Table 5:

TABLE 5
Effect of LBH589 on Median Time to Clinical Endpoint

| Treatment | N failed | N Censored | Median (days) | 95% CI |
|---|---|---|---|---|
| Vehicle | 8 | 0 | 37 | (34, 38) |
| LBH589 10 mg/kg | 8 | 0 | 54 | (49, 56) |
| LBH589 20 mg/kg | 5* | 1** | 61 | (58, —) |

*2 animals were found dead on Day 45 after implantation. Animals exhibited 15% body weight loss and abdominal distension, but no clinical symptoms of bone disease prior to death. Deaths were ruled as treatment related and removed from analysis.
**One animal exhibited no signs of disease by 80 days post implantation and was censored

Two of the eight animals treated with LBH589 at 20 mg/kg that died on Day 45 did not demonstrate signs of bone disease prior to death and were ruled as treatment related deaths. These animals were removed from analysis due to treatment related deaths. One of the remaining six animals did not exhibit any symptoms of disease 80 days after implant, when the observations were terminated and was censored in endpoint analysis. The median time to endpoint for the vehicle treated animals was 37 days. LBH589 dosed at 10 and 20 mg/kg resulted in median time to clinical endpoint of 54 and 61 days, respectively. The dose response increase in median time to achieve endpoint was significantly different, as evidenced by the non-overlapping 95% confidence intervals.

### LBH589 Effects on Trabecular Bone

MicroCT was used to evaluate the effects on trabecular bone of LBH589 in MM1S tumor bearing mice. The regions of interest and representative images are shown in FIG. 4.

FIG. 4 describes a 2.3 mm region of interest was placed to start at the growth plate extending distally. 106 axial slices were obtained using a p-CT VivaCT40 Scanner (SCANCO, Switzerland) with 55 kv, 145 mA, 180 ms integration time and 21 μm resolution. Trabecular bone density, bone volume/total volume (BV/TV), was measured in a 0.735 mm region of a tibia (10 slices proximal and 25 slices distal from the tibial tuberosity) using SCANCO software (SCANCO, Switzerland) with a threshold of 275. Cortical bone density, bone volume/total volume (BV/TV), was measured in a 1.5 mm region of a tibia (15 slices proximal and 55 slices distal from the tibial tuberosity) using SCANCO software (SCANCO Switzerland) with a threshold of 275.

The mean trabecular bone density (BV/TV) and percent change (treated as a percent of control) are shown in FIG. 5 and Tables 6 and 7.

TABLE 6
0879 Delta Mean Trabecular Density

| Treatments | Vehicle | LBH589 (15 mg/kg) |
|---|---|---|
| Number of Values | 8 | 7 |
| Mean | 2.23 | 14.48 |
| Standard Error | 0.344 | 1.97 |
| % Relative mean change | n/a | 549% |
| treated/vehicle | | |

TABLE 7
0942 Delta Median Trabecular Density

| | | LBH589 | LBH589 |
|---|---|---|---|
| Treatments | Vehicle | (10 mg/kg) | (20 mg/kg) |
| Number of Values | 8 | 8 | 8 |
| Median | 0.64 | 8.23 | 12.89 |
| % Relative median change | n/a | 1186% | 1914% |
| treated/vehicle | | | |

Statistical analysis of trabecular bone density are presented in Tables 8 and 9.

TABLE 8
0879 Statistics of Trabecular Bone Density

| Treatments | Vehicle | LBH589 (15 mg/kg) |
|---|---|---|
| Vehicle | X | S |
| LBH589 (15) | | X |

Students T-Test
S = P < 0.05

TABLE 9
0942 Statistics of Trabecular Bone Density

| Treatments (#0879) | Vehicle | LBH589 (10 mg/kg) | LBH589 (20 mg/kg) |
|---|---|---|---|
| Vehicle | X | S | S |
| LBH589 (10) | | X | S |
| LBH589 (20) | | | X |

Wilcoxon/Kruskal-Wallis Test with Tukey-Kramer Post-hoc multiple comparison
S = P < 0.05

LBH589 at 15 mg/kg resulted in a statistically significant 5.5-fold increase in mean trabecular bone density after 3 weeks of treatment. In Study #0942, LBH589 dosed at 10 and 20 mg/kg resulted in a statistically significant increase in median trabecular bone density of 11.8- and 19.1-fold, respectively, after 4 weeks of treatment.

FIG. 5 describes that trabecular bone density (% BV/TV) was analyzed. In panel A and B, the bar graphs represent the mean average t SEM of the tibial trabecular bone density (% BV/TV) for Study #0879 and #0942. The right panels are the results of individual animals are represented in the graphs above. * indicates statistical significance from vehicle control at the same time point (p<0.05).

### LBH589 Effects on Cortical Bone

The effects on cortical bone of LBH589 as a single agent was evaluated by microCT analysis in Study #0879. Quantitative analysis of the cortical bone density and their relative differences for Study #0879 are represented in FIG. 6 and Table 10.

TABLE 10
0879 Delta Mean Cortical Density

| Treatments | Vehicle | LBH589 (15 mg/kg) |
|---|---|---|
| Number of values | 8 | 7 |
| Mean | 88.7% | 98.3% |
| Standard error | 1.4% | 0.4% |
| % Relative mean change | N/A | 10.8% |
| treated/vehicle | | |

Statistical analysis is presented in Table 11. Treatment with LBH589 resulted in statistically significant 10.8% increase in cortical bone density relative to vehicle treated animals.

TABLE 11
0879 Statistics of Cortical Bone Density

| Treatments | Vehicle | LBH589 (15 mg/kg) |
|---|---|---|
| Vehicle | X | S |
| LBH589 (15 mg/kg) | | X |

Students T-Test
S = P < 0.0001

### Serum Biomarker Evaluation

TRACP5B serum levels were evaluated as a measure of osteoclast activity. The level of a TRACP5B was analyzed in FIG. 7 and Table 12.

TABLE 12
0879 Delta Mean\TRACP5B Serum Levels

| Treatments | Vehicle | LBH589 (15 mg/kg) |
|---|---|---|
| Number of Values | 8 | 5 |
| Mean | 12.0 | 7.1 |
| Standard Error | 1.3 | 1.1 |
| % Relative mean change | n/a. | −41% |
| treated/vehicle | | |

Serum bio-marker TRACP5b was analyzed as described in the Methods. In the left panel, the bar graph represents the mean average±SEM. * indicates statistical significance from controls (p<0.05).

Serum levels of TRACP5B in this study were significantly decreased by 41% in animals treated with LBH589 alone as compared to vehicle treated animals, Table 13:

TABLE 13
0879 Statistics of TRACP5B Serum Levels

| Treatments | Vehicle | LBH589 (15 mg/kg) |
|---|---|---|
| Vehicle | X | S |
| LBH589 (15) | | X |

Students T-Test
S = P < 0.05

1. A method of treating a patient suffering from bone destruction cause by a proliferative disease comprising administering to the patient an effective amount of a HDAC inhibitor.

2. The method of claim 1, wherein the proliferative disease is selected from multiple myeloma, breast cancer, or prostate cancer.

3. The method of claim 1, wherein the HDAC inhibitor is wherein the HDAC inhibitor is a compound of the formula (I): 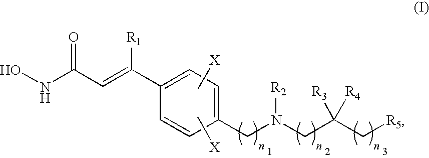
wherein
R₁ is H; halo; or a straight-chain C₁-C₆alkyl, especially methyl, ethyl or n-propyl, which methyl, ethyl and n-propyl substituents are unsubstituted or substituted by one or more substituents described below for alkyl substituents;
R₂ is selected from H; C₁-C₁₀alkyl, preferably C₁-C₈alkyl, e.g., methyl, ethyl or —CH₂CH₂—OH; C₄-C₉cycloalkyl; C₄-C₉heterocycloalkyl; C₄-C₈heterocycloalkylalkyl; cycloalkylalkyl, e.g., cyclopropylmethyl; aryl; heteroaryl; arylalkyl, e.g., benzyl; heteroarylalkyl, e.g., pyridylmethyl; —(CH₂)ₙC(O)R₆; —(CH₂)ₙOC(O)R₆; amino acyl; HON—C(O)—CH═C(R₁)-aryl-alkyl-; and —(CH₂)ₙR₇;
R₃ and R₄ are the same or different and, independently, H, C₁-C₈alkyl, acyl or acylamino, or
R₃ and R₄, together with the carbon to which they are bound, represent C═O, C═S or C═NR₈, or
R₂, together with the nitrogen to which it is bound, and R₃, together with the carbon to which it is bound, can form a C₄-C₉heterocycloalkyl, a heteroaryl, a polyheteroaryl, a non-aromatic polyheterocycle, or a mixed aryl and non-aryl polyheterocycle ring;
R₅ is selected from H; C₁-C₆alkyl; C₄-C₉cycloalkyl; C₄-C₉heterocycloalkyl; acyl; aryl; heteroaryl; arylalkyl, e.g., benzyl; heteroarylalkyl, e.g., pyridylmethyl; aromatic polycycles; non-aromatic polycycles; mixed aryl and non-aryl polycycles; polyheteroaryl; non-aromatic polyheterocycles; and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H; halo; C₁-C₄alkyl, such as CH₃ and CF₃; NO₂; C(O)R₁; OR₉; SR₉; CN; and NR₁₀R₁₁;
R₆ is selected from H; C₁-C₆alkyl; C₄-C₉cycloalkyl; C₄-C₉heterocycloalkyl; cycloalkylalkyl, e.g., cyclopropylmethyl; aryl; heteroaryl; arylalkyl, e.g., benzyl and 2-phenylethenyl; heteroarylalkyl, e.g., pyridylmethyl; OR₁₂; and NR₁₃R₁₄;
R₇ is selected from OR₁₅, SR₁₅, S(O)R₁₆, SO₂R₁₇, NR₁₃R₁₄ and NR₁₂SO₂R₆,
R₈ is selected from H; OR₁₅; NR₁₃R₁₄; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
R₉ is selected from C₁-C₄alkyl, e.g., CH₃ and CF₃; C(O)-alkyl, e.g., C(O)CH₃; and C(O)CF₃;
R₁₀ and R₁₁ are the same or different and independently selected from H, C₁-C₄alkyl and —C(O)-alkyl;
R₁₂ is selected from H; C₁-C₆alkyl; C₄-C₉cycloalkyl; C₄-C₉heterocycloalkyl; C₄-C₉heterocycloalkylalkyl; aryl; mixed aryl and non-aryl polycycle; heteroaryl; arylalkyl, e.g., benzyl; and heteroarylalkyl, e.g., pyridylmethyl;
R₁₃ and R₁₄ are the same or different and independently selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl; amino acyl, or
R₁₃ and R₁₄, together with the nitrogen to which they are bound, are C₄-C₉heterocycloalkyl, heteroaryl, polyheteroaryl, non-aromatic polyheterocycle, or mixed aryl and non-aryl polyheterocycle;
R₁₅ is selected from H, C₁-C₈cycloalkyl, C₄-C₉heterocycloalkyl, aryl, heteroaryl, arylalkyl, heteroarylalkyl and (CH₂)ₘZR₁₂;
R₁₆ is selected from C₁-C₆alkyl, C₄-C₉cycloalkyl, C₄-C₉heterocycloalkyl, aryl, heteroaryl, polyheteroaryl, arylalkyl, heteroarylalkyl and (CH₂)ₘZR₁₂;
R₁₇ is selected from C₁-C₆alkyl, C₄-C₉cycloalkyl, C₄-C₉heterocycloalkyl, aryl, aromatic polycycles, heteroaryl, arylalkyl, heteroarylalkyl, polyheteroaryl and NR₁₃R₁₄;
m is an integer selected from 0-6; and
Z is selected from O, NR₁₃, S and S(O);
or a pharmaceutically acceptable salt thereof.

4. The method according to claim 1, wherein the HDAC inhibitor is N-hydroxy-3-[4-[[[2-(2-methyl-1H-indol-3-yl)-ethyl]-amino]methyl]phenyl]-2E-2-propenamide having the formula (III): 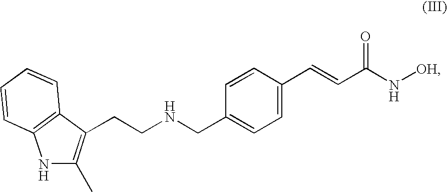
or a pharmaceutically acceptable salt thereof.

5. A method of treating a patient suffering from bone destruction cause by an inflammatory disease comprising administering to the patient an effective amount of a HDAC inhibitor.

6. The method of claim 5, wherein the HDAC inhibitor is a compound of the formula (I): 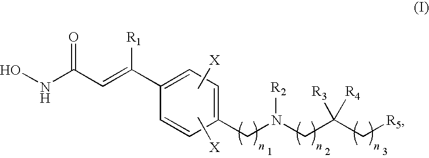
wherein
R₁ is H; halo; or a straight-chain C₁-C₆alkyl, especially methyl, ethyl or n-propyl, which methyl, ethyl and n-propyl substituents are unsubstituted or substituted by one or more substituents described below for alkyl substituents;
R₂ is selected from H; C₁-C₁₀alkyl, preferably C₁-C₈alkyl, e.g., methyl, ethyl or —CH₂CH₂—OH; C₄-C₉cycloalkyl; C₄-C₉heterocycloalkyl; C₄-C₉heterocycloalkylalkyl; cycloalkylalkyl, e.g., cyclopropylmethyl; aryl; heteroaryl; arylalkyl, e.g., benzyl; heteroarylalkyl, e.g., pyridylmethyl; —(CH₂)ₙC(O)R₆; —(CH₂)ₙOC(O)R₆; amino acyl; HON—C(O)—CH═C(R₁)-aryl-alkyl-; and —(CH₂)ₙR₇;
R₃ and R₄ are the same or different and, independently, H, acyl or acylamino, or
R₃ and R₄, together with the carbon to which they are bound, represent C═O, C═S or C═NR₈, or
R₂, together with the nitrogen to which it is bound, and R₃, together with the carbon to which it is bound, can form a C₄-C₉heterocycloalkyl, a heteroaryl, a polyheteroaryl, a non-aromatic polyheterocycle, or a mixed aryl and non-aryl polyheterocycle ring;
R₅ is selected from H; C₁-C₆alkyl; C₄-C₉cycloalkyl; C₄-C₉heterocycloalkyl; acyl; aryl; heteroaryl; arylalkyl, e.g., benzyl; heteroarylalkyl, e.g., pyridylmethyl; aromatic polycycles; non-aromatic polycycles; mixed aryl and non-aryl polycycles; polyheteroaryl; non-aromatic polyheterocycles; and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H; halo; C₁-C₄alkyl, such as CH₃ and CF₃; NO₂; C(O)R₁; OR₉; SR₉; CN; and NR₁₀R₁₁;
R₆ is selected from H; C₁-C₆alkyl; C₄-C₉cycloalkyl; C₄-C₉heterocycloalkyl; cycloalkylalkyl, e.g., cyclopropylmethyl; aryl; heteroaryl; arylalkyl, e.g., benzyl and 2-phenylethenyl; heteroarylalkyl, e.g., pyridylmethyl; OR₁₂; and NR₁₃R₁₄;
R₇ is selected from OR₁₅, SR₁₅, S(O)R₁₆, SO₂R₁₇, NR₁₃R₁₄ and NR₁₂SO₂R₆;
R₈ is selected from H; OR₁₅; NR₁₃R₁₄, C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
R₉ is selected from C₁-C₄alkyl, e.g., CH₃ and CF₃; C(O)-alkyl, e.g., C(O)CH₃; and C(O)CF₃;
R₁₀ and R₁₁ are the same or different and independently selected from H, C₁-C₄alkyl and —C(O)-alkyl;
R₁₂ is selected from H; C₁-C₆alkyl; C₄-C₉cycloalkyl; C₄-C₉heterocycloalkyl; C₄-C₉heterocycloalkylalkyl; aryl; mixed aryl and non-aryl polycycle; heteroaryl; arylalkyl, e.g., benzyl; and heteroarylalkyl, e.g., pyridylmethyl;
R₁₃ and R₁₄ are the same or different and independently selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl; amino acyl, or
R₁₃ and R₁₄, together with the nitrogen to which they are bound, are C₄-C₉heterocycloalkyl, heteroaryl, polyheteroaryl, non-aromatic polyheterocycle, or mixed aryl and non-aryl polyheterocycle;
R₁₅ is selected from H, C₄-C₉cycloalkyl, C₄-C₉heterocycloalkyl, aryl, heteroaryl, arylalkyl, heteroarylalkyl and (CH₂)ₘZR₁₂;
R₁₆ is selected from C₁-C₆alkyl, C₄-C₉cycloalkyl, C₄-C₉heterocycloalkyl, aryl, heteroaryl, polyheteroaryl, arylalkyl, heteroarylalkyl and (CH₂)ₘZR₁₂;
R₁₇ is selected from C₁-C₆alkyl, C₄-C₉cycloalkyl, C₄-C₉heterocycloalkyl, aryl, aromatic polycycles, heteroaryl, arylalkyl, heteroarylalkyl, polyheteroaryl and NR₁₃R₁₄;
m is an integer selected from 0-6; and
Z is selected from O, NR₁₃, S and S(O);
or a pharmaceutically acceptable salt thereof.

7. The method according to claim 5, wherein the HDAC inhibitor is N-hydroxy-3-[4-[[[2-(2-methyl-1H-indol-3-yl)-ethyl]-amino]methyl]phenyl]-2E-2-propenamide having the formula (III): 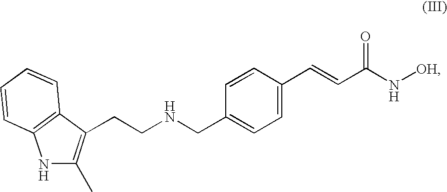
or a pharmaceutically acceptable salt thereof.

8. A method of treating a patient suffering from bone destruction cause by osteoporosis comprising administering to the patient an effective amount of a HDAC inhibitor.

9. The method of claim 8, wherein the HDAC inhibitor is wherein the HDAC inhibitor is a compound of the formula (I): 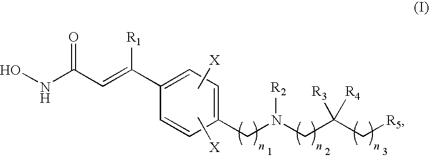
wherein
R₁ is H; halo; or a straight-chain C₁-C₆alkyl, especially methyl, ethyl or n-propyl, which methyl, ethyl and n-propyl substituents are unsubstituted or substituted by one or more substituents described below for alkyl substituents;
R₂ is selected from H; C₁-C₁₀alkyl, preferably C₁-C₆alkyl, e.g., methyl, ethyl or —CH₂CH₂—OH; C₄-C₉cycloalkyl; C₄-C₉heterocycloalkyl; C₄-C₉heterocycloalkylalkyl; cycloalkylalkyl, e.g., cyclopropylmethyl; aryl; heteroaryl; arylalkyl, e.g., benzyl; heteroarylalkyl, e.g., pyridylmethyl; —(CH₂)ₙC(O)R₆; —(CH₂)ₙOC(O)R₆; amino acyl; HON—C(O)—CH═C(R₁)-aryl-alkyl-; and —(CH₂)ₙR₇,
R₃ and R₄ are the same or different and, independently, H, acyl or acylamino, or
R₃ and R₄, together with the carbon to which they are bound, represent C═O, C═S or C═NR₈, or
R₂, together with the nitrogen to which it is bound, and R₃, together with the carbon to which it is bound, can form a C₄-C₈heterocycloalkyl, a heteroaryl, a polyheteroaryl, a non-aromatic polyheterocycle; or a mixed aryl and non-aryl polyheterocycle ring;
R₅ is selected from H; C₁-C₆alkyl; C₄-C₉cycloalkyl; C₄-C₉heterocycloalkyl; acyl; aryl; heteroaryl; arylalkyl, e.g., benzyl; heteroarylalkyl, e.g., pyridylmethyl; aromatic polycycles; non-aromatic polycycles; mixed aryl and non-aryl polycycles; polyheteroaryl; non-aromatic polyheterocycles; and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H; halo; C₁-C₄alkyl, such as CH₃ and CF₃; NO₂; C(O)R₁; OR₉; SR₉; CN; and NR₁₀R₁₁;
R₆ is selected from H; C₁-C₆alkyl; C₄-C₉cycloalkyl; C₄-C₉heterocycloalkyl; cycloalkylalkyl, e.g., cyclopropylmethyl; aryl; heteroaryl; arylalkyl, e.g., benzyl and 2-phenylethenyl; heteroarylalkyl, e.g., pyridylmethyl; OR₁₂; and NR₁₃R₁₄;
R₇ is selected from OR₁₅, SR₁₅, S(O)R₁₆, SO₂R₁₇, NR₁₃R₁₄ and NR₁₂SO₂R₆;
R₈ is selected from H; OR₁₅; NR₁₃R₁₄; C₁-C₆alkyl; C₄-C₉cycloalkyl; C₄-C₈heterocycloalkyl; aryl; heteroaryl; arylalkyl, e.g., benzyl; and heteroarylalkyl, e.g., pyridylmethyl;
R₉ is selected from C₁-C₄alkyl, e.g., CH₃ and CF₃; C(O)-alkyl, e.g., C(O)CH₃; and C(O)CF₃;
R₁₀ and R₁₁ are the same or different and independently selected from H, C₁-C₄alkyl and —C(O)-alkyl;
R₁₂ is selected from H; C₁-C₆alkyl; C₄-C₉cycloalkyl; C₄-C₈heterocycloalkyl; C₄-C₉heterocycloalkylalkyl; aryl; mixed aryl and non-aryl polycycle; heteroaryl; arylalkyl, e.g., benzyl; and heteroarylalkyl, e.g., pyridylmethyl;
R₁₃ and R₁₄ are the same or different and independently selected from H; C₁-C₈alkyl; C₄-C₉cycloalkyl; C₄-C₉heterocycloalkyl; aryl; heteroaryl; arylalkyl, e.g., benzyl; heteroarylalkyl, e.g., pyridylmethyl; amino acyl, or
R₁₃ and R₁₄, together with the nitrogen to which they are bound, are C₄-C₈heterocycloalkyl, heteroaryl, polyheteroaryl, non-aromatic polyheterocycle, or mixed aryl and non-aryl polyheterocycle;
R₁₅ is selected from H, C₁-C₆alkyl, C₄-C₉cycloalkyl, C₄-C₉heterocycloalkyl, aryl, heteroaryl, arylalkyl, heteroarylalkyl and (CH₂)ₘZR₁₂,
R₁₆ is selected from C₁-C₆alkyl, C₄-C₉cycloalkyl, C₄-C₉heterocycloalkyl, aryl, heteroaryl, polyheteroaryl, arylalkyl, heteroarylalkyl and (CH₂)ₘZR₁₂;
R₁₇ is selected from C₁-C₆alkyl, C₄-C₉cycloalkyl, C₄-C₉heterocycloalkyl, aryl, aromatic polycycles, heteroaryl, arylalkyl, heteroarylalkyl, polyheteroaryl and NR₁₃R₁₄;
m is an integer selected from 0-6; and
Z is selected from O, NR₁₃, S and S(O);
or a pharmaceutically acceptable salt thereof.

10. The method according to claim 8, wherein the HDAC inhibitor is N-hydroxy-3-[4-[[[2-(2-methyl-1H-indol-3-yl)-ethyl]-amino]methyl]phenyl]-2E-2-propenamide having the formula (III): 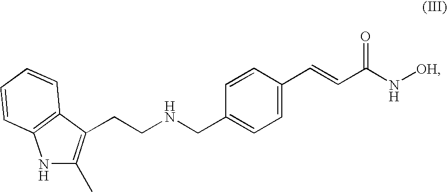
or a pharmaceutically acceptable salt thereof.

11-20. (canceled)

21. A method of preventing bone loss in a patient suffering from a proliferative disease comprising administering to said patient an effective amount of a HDAC inhibitor.

22. The method according to claim 21, wherein the proliferative disease is selected from multiple myeloma, breast cancer or prostate cancer.

23. The method according to claim 21, wherein the HDAC inhibitor is a compound of the formula (I): 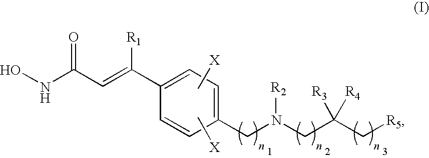
wherein
R₁ is H; halo; or a straight-chain C₁-C₆alkyl, especially methyl, ethyl or n-propyl, which methyl, ethyl and n-propyl substituents are unsubstituted or substituted by one or more substituents described below for alkyl substituents;
R₂ is selected from H; C₁-C₁₀alkyl, preferably C₁-C₈alkyl, e.g., methyl, ethyl or —CH₂CH₂—OH; C₄-C₉cycloalkyl; C₄-C₈heterocycloalkyl; C₄-C₉heterocycloalkylalkyl; cycloalkylalkyl, e.g., cyclopropylmethyl; aryl; heteroaryl; arylalkyl, e.g., benzyl; heteroarylalkyl, e.g., pyridylmethyl; —(CH₂)ₙC(O)R₈; —(CH₂)OC(O)R₈; amino acyl; HON—C(O)—CH═C(R₁)-aryl-alkyl-; and —(CH₂)ₙR₇,
R₃ and R₄ are the same or different and, independently, H, C₁-C₈alkyl, acyl or acylamino, or
R₃ and R₄, together with the carbon to which they are bound, represent C═O, C═S or C═NR₈, or
R₂, together with the nitrogen to which it is bound, and R₃, together with the carbon to which it is bound, can form a C₄-C₉heterocycloalkyl, a heteroaryl, a polyheteroaryl, a non-aromatic polyheterocycle, or a mixed aryl and non-aryl polyheterocycle ring;
R₅ is selected from H; C₁-C₈alkyl; C₄-C₉cycloalkyl; C₄-C₉heterocycloalkyl; acyl; aryl; heteroaryl; arylalkyl, e.g., benzyl; heteroarylalkyl, e.g., pyridylmethyl; aromatic polycycles; non-aromatic polycycles; mixed aryl and non-aryl polycycles; polyheteroaryl; non-aromatic polyheterocycles; and mixed aryl and non-aryl polyheterocycles;
n, nᵢ, n₂ and n₃ are the same or different and independently selected from 0-6, when n1 is 1-6, each carbon atom can be optionally and independently substituted with R₃ and/or R₄;
X and Y are the same or different and independently selected from H; halo; C₁-C₄alkyl, such as CH₃ and CF₃; NO₂; C(O)R₁; OR₉; SR₉; CN; and NR₁₀R₁₁;
R₆ is selected from H; C₁-C₆alkyl; C₄-C₉cycloalkyl; C₄-C₉heterocycloalkyl; cycloalkylalkyl, e.g., cyclopropylmethyl; aryl; heteroaryl; arylalkyl, e.g., benzyl and 2-phenylethenyl; heteroarylalkyl, e.g., pyridylmethyl; OR₁₂; and NR₁₃R₁₄;
R₇ is selected from OR₁₅, SR₁₅, S(O)R₁₆, SO₂R₁₇, NR₁₃R₁₄ and NR₁₂SO₂R₆;
R₈ is selected from H; OR₁₅; NR₁₃R₁₄; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
R₉ is selected from C₁-C₄alkyl, e.g., CH₃ and CF₃; C(O)-alkyl, e.g., C(O)CH₃; and C(O)CF₃;
R₁₀ and R₁₁ are the same or different and independently selected from H, C₁-C₄alkyl and —C(O)-alkyl;
R₁₂ is selected from H; C₁-C₈alkyl; C₄-C₉cycloalkyl; C₄-C₉heterocycloalkyl; C₄-C₉heterocycloalkylalkyl; aryl; mixed aryl and non-aryl polycycle; heteroaryl; arylalkyl, e.g., benzyl; and heteroarylalkyl, e.g., pyridylmethyl;
R₁₃ and R₁₄ are the same or different and independently selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl; amino acyl, or
R₁₃ and R₁₄, together with the nitrogen to which they are bound, are C₄-C₉heterocycloalkyl, heteroaryl, polyheteroaryl, non-aromatic polyheterocycle, or mixed aryl and non-aryl polyheterocycle;
R₁₅ is selected from H, C₁-C₆alkyl, C₄-C₉cycloalkyl, C₄-C₉heterocycloalkyl, aryl, heteroaryl, arylalkyl, heteroarylalkyl and (CH₂)ₘZR₁₂;
R₁₆ is selected from C₁-C₆alkyl, C₄-C₉cycloalkyl, C₄-C₉heterocycloalkyl, aryl, heteroaryl, polyheteroaryl, arylalkyl, heteroarylalkyl and (CH₂)ₘZR₁₂;
R₁₇ is selected from C₁-C₆alkyl, C₄-C₉cycloalkyl, C₄-C₉heterocycloalkyl, aryl, aromatic polycycles, heteroaryl, arylalkyl, heteroarylalkyl, polyheteroaryl and NR₁₃R₁₄;
m is an integer selected from 0-6; and
Z is selected from O, NR₁₃, S and S(O);
or a pharmaceutically acceptable salt thereof.

24. The method according to claim 21, wherein the HDAC inhibitor is N-hydroxy-3-[4-[[[2-(2-methyl-1H-indol-3-yl)-ethyl]-amino]methyl]phenyl]-2E-2-propenamide having the formula (III): 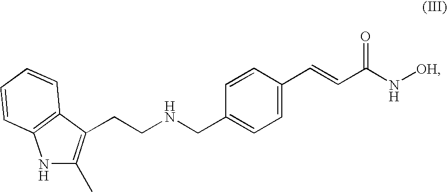
or a pharmaceutically acceptable salt thereof.

25. A method of preventing bone loss in a patient suffering from an inflammatory disease comprising administering to said patient an effective amount of a HDAC inhibitor.

26. The method according to claim 25, wherein the HDAC inhibitor is a compound of the formula (I): 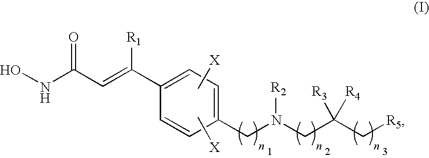
wherein
R₁ is H; halo; or a straight-chain C₁-C₈alkyl, especially methyl, ethyl or n-propyl, which methyl, ethyl and n-propyl substituents are unsubstituted or substituted by one or more substituents described below for alkyl substituents;
R₂ is selected from H; C₁-C₁₀alkyl, preferably C₁-C₈alkyl, e.g., methyl, ethyl or —CH₂CH₂—OH; C₄-C₉cycloalkyl; C₄-C₉heterocycloalkyl; C₄-C₉heterocycloalkylalkyl; cycloalkylalkyl, e.g., cyclopropylmethyl; aryl; heteroaryl; arylalkyl, e.g., benzyl; heteroarylalkyl, e.g., pyridylmethyl; —(CH₂)ₙC(O)R₆; —(CH₂)ₙOC(O)R₆; amino acyl; HON—C(O)—CH═C(R₁)-aryl-alkyl-; and —(CH₂)₉R₇;
R₃ and R₄ are the same or different and, independently, H, acyl or acylamino, or
R₃ and R₄, together with the carbon to which they are bound, represent C═O, C═S or C═NR₈, or
R₂, together with the nitrogen to which it is bound, and R₃, together with the carbon to which it is bound, can form a C₄-C₉heterocycloalkyl, a heteroaryl, a polyheteroaryl, a non-aromatic polyheterocycle, or a mixed aryl and non-aryl polyheterocycle ring;
R₅ is selected from H; C₁-C₆alkyl; C₄-C₉cycloalkyl; C₄-C₉heterocycloalkyl; acyl; aryl; heteroaryl; arylalkyl, e.g., benzyl; heteroarylalkyl, e.g., pyridylmethyl; aromatic polycycles; non-aromatic polycycles; mixed aryl and non-aryl polycycles; polyheteroaryl; non-aromatic polyheterocycles; and mixed aryl and non-aryl polyheterocycles;
n, n₁, n₂ and n₃ are the same or different and independently selected from 0-6, when n, is 1-6, each carbon atom can be optionally and independently substituted with R₃ and/or R₄;
X and Y are the same or different and independently selected from H; halo; C₁-C₄alkyl, such as CH₃ and CF₃; NO₂; C(O)R₁; OR₉; SR₉; CN; and NR₁₀R₁₁;
R₆ is selected from H; C₁-C₆alkyl; C₄-C₉cycloalkyl; C₄-C₉heterocycloalkyl; cycloalkylalkyl, e.g., cyclopropylmethyl; aryl; heteroaryl; arylalkyl, e.g., benzyl and 2-phenylethenyl; heteroarylalkyl, e.g., pyridylmethyl; OR₁₂; and NR₁₃R₁₄;
R₇ is selected from OR₁₅, SR₁₅, S(O)R₁₆, SO₂R₁₇, NR₁₃R₁₄ and NR₁₂SO₂R₆;
R₈ is selected from H; OR₁₅; NR₁₃R₁₄; C₁-C₈alkyl; C₄-C₉cycloalkyl; C₄-C₉heterocycloalkyl; aryl; heteroaryl; arylalkyl, e.g., benzyl; and heteroarylalkyl, e.g., pyridylmethyl;
R₉ is selected from C₁-C₄alkyl, e.g., CH₃ and CF₃; C(O)-alkyl, e.g., C(O)CH₃; and C(O)CF₃;
R₁₀ and R₁₁ are the same or different and independently selected from H, C₁-C₄alkyl and —C(O)-alkyl;
R₁₂ is selected from H; C₁-C₆alkyl; C₄-C₉cycloalkyl; C₄-C₉heterocycloalkyl; C₄-C₉heterocycloalkylalkyl; aryl; mixed aryl and non-aryl polycycle; heteroaryl; arylalkyl, e.g., benzyl; and heteroarylalkyl, e.g., pyridylmethyl;
R₁₃ and R₁₄ are the same or different and independently selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl; amino acyl, or
R₁₃ and R₁₄, together with the nitrogen to which they are bound, are C₄-C₉heterocycloalkyl, heteroaryl, polyheteroaryl, non-aromatic polyheterocycle, or mixed aryl and non-aryl polyheterocycle;
R₁₅ is selected from H, C₁-C₆alkyl, C₄-C₉cycloalkyl, C₄-C₉heterocycloalkyl, aryl, heteroaryl, arylalkyl, heteroarylalkyl and (CH₂)ₘZR₁₂;
R₁₆ is selected from C₁-C₆alkyl, C₄-C₉cycloalkyl, C₄-C₉heterocycloalkyl, aryl, heteroaryl, polyheteroaryl, arylalkyl, heteroarylalkyl and (CH₂)ₘZR₁₂;
R₁₇ is selected from C₁-C₆alkyl, C₄-C₉cycloalkyl, C₄-C₉heterocycloalkyl, aryl, aromatic polycycles, heteroaryl, arylalkyl, heteroarylalkyl, polyheteroaryl and NR₁₃R₁₄;
m is an integer selected from 0-6; and
Z is selected from O, NR₁₃, S and S(O);
or a pharmaceutically acceptable salt thereof.

27. The method according to claim 25, wherein the HDAC inhibitor is N-hydroxy-3-[4-[[[2-(2-methyl-1H-indol-3-yl)-ethyl]-amino]methyl]phenyl]-2E-2-propenamide having the formula (III): 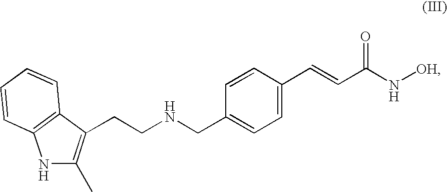
or a pharmaceutically acceptable salt thereof.

28. A method of preventing bone loss in a patient suffering from osteoporosis comprising administering to said patient an effective amount of a HDAC inhibitor.

29. The method according to claim 28, wherein the HDAC inhibitor is a compound of the formula (I): 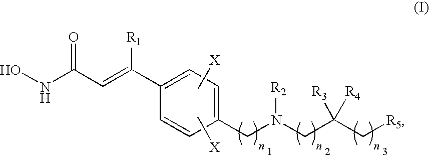
wherein
R₁ is H; halo; or a straight-chain C₁-C₆alkyl, especially methyl, ethyl or n-propyl, which methyl, ethyl and n-propyl substituents are unsubstituted or substituted by one or more substituents described below for alkyl substituents;
R₂ is selected from H; C₁-C₁₀alkyl, preferably C₁-C₆alkyl, e.g., methyl, ethyl or —CH₂CH₂—OH; C₄-C₉cycloalkyl; C₄-C₉heterocycloalkyl; C₄-C₉heterocycloalkylalkyl; cycloalkylalkyl, e.g., cyclopropylmethyl; aryl; heteroaryl; arylalkyl, e.g., benzyl; heteroarylalkyl, e.g., pyridylmethyl; —(CH₂)ₙC(O)R₆; —(CH₂)ₙOC(O)R₆; amino acyl; HON—C(O)—CH═C(R₁)-aryl-alkyl-; and —(CH₂)ₙR₇;
R₃ and R₄ are the same or different and, independently, H, C₁-C₆alkyl, acyl or acylamino, or
R₃ and R₄, together with the carbon to which they are bound, represent C═O, C═S or C═NR₆, or
R₂, together with the nitrogen to which it is bound, and R₃, together with the carbon to which it is bound, can form a C₄-C₉heterocycloalkyl, a heteroaryl, a polyheteroaryl, a non-aromatic polyheterocycle, or a mixed aryl and non-aryl polyheterocycle ring;
R₅ is selected from H; C₁-C₆alkyl; C₄-C₉cycloalkyl; C₄-C₉heterocycloalkyl; acyl; aryl; heteroaryl; arylalkyl, e.g., benzyl; heteroarylalkyl, e.g., pyridylmethyl; aromatic polycycles; non-aromatic polycycles; mixed aryl and non-aryl polycycles; polyheteroaryl; non-aromatic polyheterocycles; and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H; halo; C₁-C₄alkyl, such as CH₃ and CF₃; NO₂; C(O)R₁; OR₉; SR₉; CN; and NR₁₀R₁₁;
R₆ is selected from H; C₁-C₆alkyl; C₄-C₉cycloalkyl; C₄-C₉heterocycloalkyl; cycloalkylalkyl, e.g., cyclopropylmethyl; aryl; heteroaryl; arylalkyl, e.g., benzyl and 2-phenylethenyl; heteroarylalkyl, e.g., pyridylmethyl; OR₁₂; and NR₁₃R₁₄;
R₇ is selected from OR₁₅, SR₁₅, S(O)R₁₆, SO₂R₁₇, NR₁₃R₁₄ and NR₁₂SO₂R₆;
R₈ is selected from H; OR₁₅; NR₁₃R₁₄; C₁-C₆alkyl; C₄-C₉cycloalkyl; C₄-C₉heterocycloalkyl; aryl; heteroaryl; arylalkyl, e.g., benzyl; and heteroarylalkyl, e.g., pyridylmethyl;
R₉ is selected from C₁-C₄alkyl, e.g., CH₃ and CF₃; C(O)-alkyl, e.g., C(O)CH₃; and C(O)CF₃;
R₁₀ and R₁₁ are the same or different and independently selected from H, C₁-C₄alkyl and —C(O)-alkyl;
R₁₂ is selected from H; C₁-C₆alkyl; C₄-C₉cycloalkyl; C₄-C₉heterocycloalkyl; C₄-C₉heterocycloalkylalkyl; aryl; mixed aryl and non-aryl polycycle; heteroaryl; arylalkyl, e.g., benzyl; and heteroarylalkyl, e.g., pyridylmethyl;
R₁₃ and R₁₄ are the same or different and independently selected from H; C₁-C₆alkyl; C₄-C₉cycloalkyl; C₄-C₉heterocycloalkyl; aryl; heteroaryl; arylalkyl, e.g., benzyl; heteroarylalkyl, e.g., pyridylmethyl; amino acyl, or
R₁₃ and R₁₄, together with the nitrogen to which they are bound, are C₄-C₉heterocycloalkyl, heteroaryl, polyheteroaryl, non-aromatic polyheterocycle, or mixed aryl and non-aryl polyheterocycle;
R₁₅ is selected from H, C₁-C₆alkyl, C₄-C₉cycloalkyl, C₄-C₉heterocycloalkyl, aryl, heteroaryl, arylalkyl, heteroarylalkyl and (CH₂)ₘZR₁₂;
R₁₆ is selected from C₁-C₆alkyl, C₄-C₉cycloalkyl, C₄-C₉heterocycloalkyl, aryl, heteroaryl, polyheteroaryl, arylalkyl, heteroarylalkyl and (CH₂)ₘZR₁₂,
R₁₇ is selected from C₁-C₆alkyl, C₄-C₉cycloalkyl, C₄-C₉heterocycloalkyl, aryl, aromatic polycycles, heteroaryl, arylalkyl, heteroarylalkyl, polyheteroaryl and NR₁₃R₁₄;
m is an integer selected from 0-6; and
Z is selected from O, NR₁₃, S and S(O);
or a pharmaceutically acceptable salt thereof.

30. The method according to claim 28, wherein the HDAC inhibitor is N-hydroxy-3-[4-[[[2-(2-methyl-1H-indol-3-yl)ethyl]-amino]methyl]phenyl]-2E-2-propenamide having the formula (III): 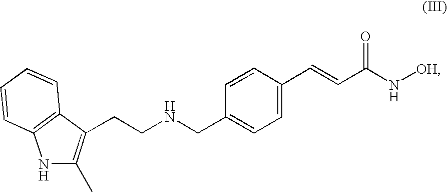
or a pharmaceutically acceptable salt thereof.
